(19) United States
(12) Patent Application Publication
(10) Pub. No.: US 2006/0025335 A1
(43) Pub. Date: Feb. 2, 2006

(54) Netrin compositions and methods of using the same

(76) Inventors: Thomas B. Kinane, Wellesley, MA (US); Katsumi Komatsuzaki, Belmont, MA (US); Ngoc Ly, Chestnut Hill, MA (US)

Correspondence Address: EDWARDS & ANGELL, LLP, P.O. BOX 55874, BOSTON, MA, 02205, US

(21) Appl. No.: 11/046,653

(22) Filed: Jan. 28, 2005

Related U.S. Application Data
(60) Provisional application No. 60/540,807, filed on Jan. 30, 2004.
(60) Provisional application No. 60/571,953, filed on May 17, 2004.

(51) Int. Cl. A61K 38/17

(52) U.S. Cl. 514/12

(57) ABSTRACT

The present invention provides methods and compositions to modulate inflammation and inflammatory responses using Netrin polypeptides and Netrin receptors. Methods of the present invention comprise the use of Netrin polypeptides and Netrin receptors to decrease migration of inflammatory cells of the immune system to a site of injury or infection.

### RELATED APPLICATIONS/PATENTS & INCORPORATION BY REFERENCE

This application claims priority to U.S. Provisional Application Ser. No. 60/540,807, filed on Jan. 30, 2004 as Attorney Docket No. 910000-3062 and U.S. Provisional Application Ser. No. 60/571,953, filed on May 17, 2004 as Attorney Docket No. 910000-3062.1, the contents each of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by a National Institutes of Health Grant No. HL058819-04. The government may have certain rights to the invention.

### BACKGROUND OF THE INVENTION

Inflammatory reactions can provide an essential defense to infection and injury. At the same time, many human diseases are characterized or aggravated by excessive inflammatory reactions. Presently, development of specific and efficacious anti-inflammatory treatment modalities is challenging. For example, use of pharmacologic anti-inflammatory agents (e.g., corticosteroids, NSAIDs, and cytokine antagonists) is frequently complicated by adverse events limiting their clinical utility. Therefore, identification of biocompatible agents that reduce inflammation has important therapeutic implications.

Endogenous proteins, such as chemokines, are viable candidates for use in improved anti-inflammatory therapies. Chemokines provide the directional cues for the migration of inflammatory cells in development, homeostasis, and responses to infection and inflammation. They play an important role in recruiting, for example, T cells, B cells and dendritic cells in organized lymphoid tissues to generate an immune response, followed by directing these effector cells into sites of infection or inflammation.

Chemokines comprise a superfamily of secreted chemotactic proteins that mediate the attraction of cells to sites of infection and inflammation. Typically, chemokines are secreted basic proteins that range from about 8 to about 10 kDa, share about 20 to about 70 percent homology in structure, and have the common functional activity of being chemotactic for inflammatory cells (Luster, A. D., New Engl. J. Med. 338: 436-45). Over 40 chemokines have been identified to date, and are subdivided into families based on the relative position of their cysteine residues. There are at least four families of chemokines, including the α and β chemokines, which contain four cysteines. The α, or cysteine-X-amino acid-cysteine (CXC) chemokines have their first two cysteine residues separated by one amino acid, whereas the first two cysteine residues of the β, or cysteine-cysteine (CC) chemokines are adjacent to each other.

Chemokines signal inflammatory cells through a sub-family of seven transmembrane spanning G-protein-coupled receptors (GPCR) (Murphy, P. M., Ann. Rev. Immunol. 12: 593-633). Chemokine receptors are named based on the subfamily of chemokines with which they principally interact. Although most chemokine receptors are capable of binding more than one chemokine, chemokine receptors generally show specificity of ligand binding in vivo.

Certain cell types, such as neurons, utilize small secreted proteins to regulate migration. The balance between chemoattractant and chemorepellent signals has been well characterized for axonal migration in neurons. In the developing brain, Netrin 1 is a key factor that induces repulsion, or abolishes chemoattraction or attracts axons and neurons depending on which receptor it binds. Netrin 1 is a secreted 63 kDa protein, which is homologous to laminin in its N-terminal domain, and contains several EGF-type repeats in its C-terminus (Serafini, T., Cell 78: 409-24). Netrin 1 is expressed during development by midline structures and attracts motor neurons and commissural axons in Drosophila (Harris, R., Neuron 17: 217-28) and in mice (Serafini, T., Cell 78: 409-24; Serafini, T., Cell 87: 1001-14). Netrin proteins are also expressed in other parts of the brain (Livesey, F. J., et al., Mol. Cell. Neurosci. 8: 417-29), as well as in embryonic heart, lung and gut (Kennedy, T. E., et al., Cell 78: 425-35).

Two types of Netrin-binding receptors have been identified: DCC and UNC5. Both receptors consist of a ligand binding extracellular domain, a single transmembrane region and a cytoplasmic tail capable of initiating downstream signaling (Hedrick, L., et al., Genes Dev. 8: 1174-83). DCC, a 160-kDa protein, belongs to the immunoglobulin (Ig) cell adhesion molecules family (CAM). Netrin 1 has been shown to bind directly and specifically to DCC, and possibly its closely related receptor, neogenin. Binding of Netrin to DCC leads to multimerization of the receptor and initiation of signal transduction via its cytoplasmic domain (Stein, E., et al., Science 291: 1976-82). This binding is necessary for axonal attraction by Netrin 1 in Xenopus and also for outgrowth of rat neurons (Stein, E., et al., Science 291: 1976-82).

In addition to DCC, Netrin is also capable of binding to UNC5. To date, four UNC5 homologs, UNC5 1-4, have been isolated (Engelkamp, D., Mech. Dev. 118: 191-7; Hong, K., et al., Cell 97: 927-41). The UNC5 proteins are transmembrane receptors with a common structure consisting of two Ig and two thrombospondin domains in the extracellular region and a ZU-5 and a death domain in the cytoplasmic tail (Hong, K., et al., Cell 97: 927-41; Leung-Hagesteijn, C., et al., Cell 71: 289-99). Most experimental evidence suggests that UNC5 acts as a coreceptor of DCC. In C. elegans, Xenopus and mammals, both receptors appear to be necessary to mediate repulsion, suggesting that the role of DCC in this pathway may be to amplify the effect of UNC5 activation. However, expression of UNC5, without DCC as a coreceptor, has been demonstrated to mediate guidance responses (Keleman, K., et al., Neuron 32: 605-17). Axons expressing only UNC5 were repulsed by Netrin at a short range, but long-range repulsion could only be mediated in cells co-expressing both UNC5 and DCC Keleman, K., et al., Neuron 32: 605-17).

As with neurons, signals that prevent inappropriate migration, or quench an existing migration, may modulate inflammatory cells, but to date, such signals are not well characterized. In particular, it was heretofore unknown whether Netrins function in the immune system as in the neural system. Evidence to date suggested that the Netrin migratory function was limited to neuronal development. For example, the Netrin 1 receptor DCC—through which Netrin 1 mediates its function in neuronal cells—was thought to act without Netrin 1 in the inflammatory cells where its expression was detected. Effects mediated through DCC in inflammatory cells were not attributed to Netrin 1. See Teyssier, J. R., et al., (2001) Biochem. Biophys. Res. Commun. 283: 1031-1036. Netrins were not expected to be viable therapeutic candidates for reducing inflammation.

### SUMMARY OF THE INVENTION

It has now been demonstrated that Netrin polypeptide(s) function in the immune system by modulating inflammatory cell migration. Compositions comprising a Netrin polypeptide and methods of using such compositions can be provided to decrease inflammatory responses to, for example, infection, injury, allergy and/or transplantation, and provide a significant improvement over current methodologies and therapies used to regulate inflammation.

Thus, the present invention generally relates to new methods and compositions to modulate inflammation and inflammatory responses using Netrin polypeptides. Methods of the present invention further comprise the use of Netrin polypeptides to decrease migration of inflammatory cells of the immune system to a target site, such as a site of injury or infection.

In a specific embodiment, the site of injury or infection is a wound.

One aspect of the invention provides a method of modulating inflammatory cells in a subject, comprising contacting an inflammatory cell undergoing or likely to undergo movement with a Netrin polypeptide in an amount effective to decrease movement of the inflammatory cells to a target site in the subject, thereby modulating inflammatory cells in the subject.

In another aspect, the invention provides a method of decreasing inflammatory cell chemotaxis, comprising contacting inflammatory cells undergoing or likely to undergo chemotaxis with a Netrin polypeptide in an amount effective to decrease chemotaxic signaling in the inflammatory cells, thereby decreasing inflammatory cell chemotaxis. The chemotactic signaling inhibited by methods of the invention can comprise G-protein coupled receptor signaling.

In yet another aspect, the invention provides a method of treating inflamed tissues in a subject, comprising administering to a subject having at least one inflamed tissue a Netrin polypeptide that decreases inflammatory cell movement in an amount effective to decrease accumulation of inflammatory cells in the inflamed tissue, thereby treating the inflamed tissues in the subject.

In one embodiment, the Netrin polypeptide, or polypeptide fragment, comprises Netrin 1. Netrin polypeptides of the invention can be recombinant polypeptides, or fragments thereof.

In yet another embodiment, methods of the invention further comprise obtaining the Netrin polypeptide.

In another embodiment, the inflammatory cells comprise leukocytes, lymphocytes, natural killer cells, antigen-presenting cells and endothelial cells. Leukocytes can comprise neutrophils, basophils, mast cells, eosinophils, monocytes, and macrophages. Lymphocytes can comprise B-lymphocytes and T-lymphocytes. The antigen-presenting cells can comprise dendritic cells and stromal cells.

In yet another embodiment, the contacting occurs in vivo in a subject having or at risk of having an adverse immune response comprising inflammatory cell movement to a target site. Preferably, the subject is a human.

In yet another embodiment, the adverse immune response is an inflammatory response. The inflammatory response can be attributed to various diseases and conditions that affect one or more organs or organ systems including, but not limited to, the peripheral nervous system, the central nervous system, skin, appendix, GI tract (including but not limited to esophagus, duodenum, and colon), respiratory/pulmonary system (including but not limited to lung, nose, pharynx, larynx), eye, genitalia/reproductive system, gums, liver/biliary ductal system, renal system (including but not limited to kidneys, urinary tract, bladder), connective tissue (including but not limited to joints, cartilage), cardiovascular system, breast, lymphatic system, muscle, ear, endocrine/exocrine system (including but not limited to lacrimal glands, salivary glands, thyroid gland, pancreas), and bone/skeletal system. The immune response can be an inflammatory response associated with wound formation in any tissue, including but not limited to those mentioned herein.

In yet another embodiment, the adverse immune response is an autoimmune response. The autoimmune response can be, but is not limited to, acquired factor VIII deficiency, acquired generalized lipodystrophy, alopecia greata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyly-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

In yet another aspect, the invention provides a kit comprising a Netrin polypeptide.

In one embodiment, the invention provides a kit comprising a Netrin polypeptide for modulating inflammatory cell movement in a subject comprising a Netrin polypeptide that decreases inflammatory cell movement and instructions for using the Netrin polypeptide to modulate inflammatory cell movement in the subject in accordance with the methods described herein.

In another embodiment, the invention provides a kit for decreasing inflammatory cell chemotaxis comprising an anti-chemotactic Netrin polypeptide and instructions for using the anti-chemotactic Netrin polypeptide to decrease chemotaxic signaling in the inflammatory cells in accordance with the methods described herein.

In yet another embodiment, the invention provides a kit for treating inflamed tissues in a subject comprising a Netrin polypeptide that decreases inflammatory cell movement and instructions for using the Netrin polypeptide to decrease accumulation of inflammatory cells in the tissue of the subject in accordance with the methods described herein.

In yet another aspect, the invention provides a method of screening compositions for Netrin functional activity comprising the steps of:
a) contacting control cells with a Netrin polypeptide and measuring a physiologic effect of the control cells;
b) contacting test cells that do not express a Netrin polypeptide with a test compound suspected of modulating inflammatory cell migration and measuring a physiologic effect of the test cells; and
c) comparing the physiologic effect of the test compound to the physiologic effect of the Netrin polypeptide to identify Netrin functional activity exhibited by the test compound.

Preferably, the test compound is a non-protein analog of a Netrin polypeptide.

In one embodiment, the physiological effect is a decrease in movement of the inflammatory cells. Such decreases in movement can be observed in vivo, in vitro and ex vivo, and can be measured by changes in inflammatory cell morphology, changes in tissue or organ morphology, changes in inflammatory cell number, changes in gene expression, changes in protein expression, changes in levels of reactive oxygen species, changes in calcium levels, or changes in cAMP levels. Changes in inflammatory cell morphology can be measured by microscopy or immunocytochemistry. Changes in inflammatory cell number can be measured by flow cytometry. Changes in gene expression can be measured by microarray analysis, Northern blotting, in situ hybridization, or RT-PCR. Changes in protein expression can be measured by Western blotting, immunocytochemistry, colorimetric assay, or ELISA.

In yet another aspect, methods of the invention further comprise modulation of a Netrin receptor and methods for the identification of agents that modulate a Netrin receptor in inflammatory cells. Preferably, the Netrin receptor is UNC5H2.

In one embodiment, the invention provides a method for identifying an agent that modulates the activity of a Netrin receptor, the method comprising contacting the Netrin receptor with a test compound; and evaluating an activity of the Netrin receptor, wherein a change in activity relative to a reference value is an indication that the compound is an agent that modulates the receptor.

In yet another embodiment, the invention provides a kit for identifying an agent that modulates the activity of a Netrin receptor comprising a Netrin receptor and instructions for using the Netrin receptor to identify the agent in accordance with the methods described herein.

In another embodiment, the invention provides a method for identifying an agent useful in the treatment of a disorder related to Netrin receptor modulation, the method comprising contacting the Netrin receptor with a test compound; and evaluating an activity of the Netrin receptor, wherein a change in activity relative to a reference value is an indication that the test compound is an agent useful in a disorder related to Netrin receptor modulation.

In yet another embodiment, the invention provides a method for treating a subject having a disorder related to Netrin receptor modulation, the method comprising identifying an agent that selectively binds to a Netrin receptor, and administering to a subject in need of such treatment a pharmaceutical composition comprising the agent which is selective for a Netrin receptor.

In yet another embodiment, the invention provides a method for identifying a a Netrin receptor, the method comprising contacting test cells that express a Netrin receptor with a Netrin polypeptide and measuring a physiologic effect of the test cells; and contacting test cells that express a receptor suspected of modulating inflammatory cell migration with a Netrin polypeptide and measuring a physiologic effect of the test cells; and comparing the physiologic effect of the test cells to the physiologic effect of the control cells to identify a Netrin receptor; and isolating the Netrin receptor.

These and other objects of the invention will be described in further detail in connection with the detailed description of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting examples and with reference to the accompanying drawings in which:

FIG. 1A shows the results of a yeast two-hybrid screen, wherein the GTPase deficient G_{iα2}, gip2, interacts with selected proteins in a human lung cDNA library.

FIG. 1B is a graphical representation comparing the hGIL-2 clone and the human UNC5H2 protein. Abbreviations are as follows: (Ig) immunoglobulin domain; (Tsp) thrombospondin; (DB) DCC binding domain; (DD) death domain.

FIG. 2 depicts a co-immunoprecipitation experiment of hemagglutinin-tagged gip2 and Myc-tagged UNC5H2.

FIG. 3 shows co-localization of HA-tagged G_{iα2} (red) and Myc-tagged UNC5H2 (green) in transfected COS7 cells.

FIG. 4 demonstrates the binding of UNC5H2 to activated GTP-bound GST-tagged G_{iα2}.

FIG. 5 is a graph depicting UNC5H2 downregulation of G_{iα2}-mediated MAP kinase signaling. Data represents an average±SEM of radioactivity incorporated in myelin basic protein in six independent experiments.

FIG. 6 is a graph showing the effect of UNC5H2 on SSTR3-mediated inhibition of cAMP accumulation.

FIG. 7A represents Northern blot analysis of UNC5H2 mRNA levels in brain, heart, kidney, lung, small intestine, and muscle.

FIG. 7B represents Northern blot analysis of UNC5H2 mRNA levels in spleen, lymph node, peripheral blood leukocytes, thymus, bone marrow, and fetal liver.

FIG. 8 depicts UNC5H2 and DCC expression by immunocytochemistry in human leukocytes. Hoechst nuclear staining is shown in blue, DCC and UNC5H2 staining is shown in red.

FIG. 9A is a graph showing the average number of peripheral blood monocytes undergoing chemotaxis in response to fMLP both in the presence and absence of Netrin 1.

FIG. 9B is a graph showing the average number of lymphocytes undergoing chemotaxis in response to SDF-1 both in the presence and absence of Netrin 1.

FIG. 9C is a graph showing the average number of granulocytes undergoing chemotaxis in response to IL-8 both in the presence and absence of Netrin 1.

FIG. 9D is a graph depicting Netrin 1 pretreatment of cells results in inhibition of chemokine-induced chemotaxis

FIG. 10A demonstrates the recruitment of leukocytes in an in vivo model of peritonitis induced by thioglycollate in the presence and absence of Netrin 1.

FIG. 10B demonstrates the recruitment of leukocytes induced by IL-8 in the presence and absence of Netrin 1.

FIG. 11 depicts Netrin 1-mediated inhibition in leukocyte recruitment in an in vivo model of mouse peritonitis.

FIG. 12 depicts Netrin 1 tissue expression and cellular localization. Netrin 1 mRNA expression in mouse tissues was quantified by quantitative real-time RT-PCR. Results represent the average of 3 samples, normalized to GAPDH (FIG. 12 A). Immunohistochemical staining of the lung demonstrates high Netrin 1 (red) expression in this tissue, particularly in the endothelium of large blood vessels and capillaries (FIG. 12B). Colocalization of Netrin 1 (red) with the endothelial cell marker PECAM-1 (green) is seen in the merged image (yellow) (FIG. 12B).

FIG. 13 depicts modulation of Netrin 1 expression in the lung during infection. Expression of Netrin 1, IFN-γ and TNF-α mRNA in the lung was measured by quantitative real-time RT-PCR in mice infected with Staphylococcus aureus. Netrin 1 is rapidly downregulated in the lung at 6 hours post-infection, coincident with the influx of leukocytes to this site of abscess formation (FIG. 13A). Regulation of Netrin 1 mRNA showed an inverse relationship to the expression of the inflammatory cytokines IFN-γ and TNF-α. Treatment of HUVECs with TNF-α and IFN-γ reduced Netrin 1 expression. Netrin 1 mRNA was measured by quantitative RT-PCR in HUVECs treated with 10 ng/ml of TNF-α or IFN-γ for 6 hours. Netrin 1 mRNA expression relative to β-actin mRNA is shown in the left panel (FIG. 13B). Netrin 1 protein expression as measured by densitometry of immunoblots is shown in the right panel.

FIG. 14 depicts the amino acid sequences of human Netrin 1 (SEQ ID NO: 1).

FIG. 15 depicts the amino acid sequences of human Netrin 2 (SEQ ID NO: 2).

FIG. 16 depicts the amino acid sequence of human Netrin 4 (SEQ ID NO: 3).

FIG. 17 depicts the amino acid sequence of human UNC5H2 (SEQ ID NO: 4).

### DETAILED DESCRIPTION OF THE INVENTION

Definitions

A “Netrin polypeptide” refers to amino acid residues of any length encoding a Netrin or a homologous protein that exhibits Netrin 1 functional activity, as well as fragments thereof and degenerate variants thereof. As used herein, Netrin 1 functional activity comprises the ability to decrease inflammatory cell migration to a target site. The Netrin polypeptide can be linear or branched, it may comprise modified amino acids or amino acid analogs, and it may be interrupted by chemical moieties other than amino acids. The terms also encompass a polypeptide that has been modified naturally or by intervention; for example, disulfide bond formation, glycosylation, lipidation, acetylation, phosphorylation, or any other manipulation or modification, such as conjugation with a labeling or bioactive component.

Netrin polypeptides of the invention include but are not limited to Netrin 1 (SEQ ID NO:1, GenBank accession No. AAD09221), Netrin 2 (SEQ ID NO:2, GenBank accession No. AAC51246) and Netrin 4 (SEQ ID NO:3, GenBank accession No. AAP92113).

A “Netrin receptor” is a G protein-coupled receptor that binds to a Netrin polypeptide and activates a signal transduction cascade in response to such binding. Netrin receptors of the invention include but are not limited to UNC5H2 (SEQ ID NO:4, GenBank accession No. AAM95701), as well as homologs, fragments and degenerate variants thereof.

As used herein, the terms “protein” and “polypeptide” are used interchangeably.

The term “a homologue”, as used herein, refers to a protein or nucleic acid sharing a certain degree of sequence “identity” or sequence “similarity” with a given protein, or the nucleic acid encoding the given protein. The term “percent identity” refers to the percentage of residues in two sequences that are the same when aligned for maximum correspondence. Sequence “similarity” is related to sequence “identity”, but differs in that residues that are not exactly the same as each other, but that are functionally “similar” are taken into consideration.

Accordingly, a homologous nucleic acid molecule of the invention is at least 15, 20, 25, 30 or more nucleotides in length and hybridizes under stringent conditions to a nucleic acid molecule encoding the amino acid sequence of SEQ ID NO:1 or to a nucleic acid molecule encoding the amino acid sequence of SEQ ID NO:4. Preferably, the molecule hybridizes under highly stringent conditions. In other embodiments, the nucleic acid is at least 30, 300, 500, 700, 850, 950, or 2000 nucleotides in length. As used herein, the term “hybridizes under stringent conditions” is intended to describe conditions for hybridization and washing under which nucleotide sequences at least 60%, 85%, or 95% homologous to each other typically remain hybridized to each other. Hybridization conditions are known to those skilled in the art and can be found in Current Protocols in Molecular Biology, John Wiley & Sons, N.Y., 6.3 α subunit.1-6.3 α subunit.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

An “inflammatory cell” is a cell contributing to an immune response that can include, but is not limited to, smooth muscle cells, follicular dendritic cells, Langerhans cells, interstitial dendritic cells, interdigitating dendritic cells, blood and veiled dendritic cells, leukocytes, lymphocytes (B-lymphocytes and T-lymphocytes), monocytes, macrophages, foam cells, tissue-specific macrophages such as alveolar macrophages, microglia, mesangial cells, histiocytes, and Kupffer cells, neutrophils, basophils, mast cells, natural killer cells, endothelial cells, eosinophils, megakaryocytes, platelets, erythrocytes and polymorphonuclear cells (e.g., granulocytes).

The term “immune response” refers to the process whereby inflammatory cells are recruited from the blood to lymphoid as well as non-lymphoid tissues via a multifactorial process that involves distinct adhesive and activation steps. Inflammatory conditions cause the release of chemokines and other factors that, by upregulating and activating adhesion molecules on inflammatory cells, promote adhesion, morphological changes, and extravasation concurrent with chemotaxis through the tissues.

An “adverse immune response” refers to any immune response having a detrimental health effect in a subject, such as inflammation. Inflammation can be caused, for example, by pathogenic infection, irritation or disease. Inflammation can also be caused by autoimmunity, wherein a subject's own antibodies react with host tissue or in which immune effector T cells are autoreactive to endogenous self-peptides and cause destruction of tissue.

“Accumulation” of inflammatory cells refers to the build up of inflammatory cells during an immune response.

The term “inflamed tissue” can be used to describe any biological tissue that has mounted an immune response causing inflammation throughout or in a portion of the tissue.

The term “target site” can refer to regions, aggregates, or populations of cells or tissues where an adverse immune response has been mounted, that is, where inflammatory cells migrate to in response to chemotactic signals. A target site can be accessed in vitro or in vivo.

As used herein, “migration” and “movement” are used interchangeably.

“Modulation” of inflammatory cells refers to the ability to control, regulate, or activate a physiological response within the cells that ultimately changes the migratory state of the cells. For example, a migratory state can change from active to inactive in the presence of stimuli that inactivates migration. The change in migratory state can be associated with certain physiological responses, such as an increase in metabolic activity of inflammatory cells stimulated by factors such as cytokines and chemokines, but are not limited to these factors. Manifestations of inflammatory cell activation include increases in ligand uptake and receptor turnover, morphological changes in cell size and complexity, and permanent or transient changes in gene and/or protein expression.

“Chemotaxis” is the movement of a migratory cell toward a target in response to a signal produced by an agent (e.g., a cytokine).

A “cytokine” is a generic term for extracellular proteins or peptides that mediate cell-cell communication, often with the effect of altering the activation state of cells.

A “chemokine” is a specific type of cytokine with a conserved cysteine motif and which can serve as an attractant.

A “subject” is a vertebrate, preferably a mammal, more preferably a human. Mammals include, but are not limited to, humans, farm animals, sport animals, and pets.

The term “obtaining” as in “obtaining the Netrin or Netrin receptor” is intended to include purchasing, synthesizing or otherwise acquiring the Netrin or Netrin receptor (or indicated substance or material).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Methods of the Invention

The invention is premised on the observation that Netrin polypeptides of the invention can modulate inflammatory cell movement. Thus, compositions comprising Netrin polypeptides and methods of using such compositions can be used to decrease inflammatory cell chemotaxis.

Inflammatory Disorders

The immune response can be adverse, and can be described as either an inflammatory response or an autoimmune response, but it is not so limited. The inflammatory response can be attributed to various diseases and conditions that affect one or more organs or organ systems including, but not lmited to, the peripheral nervous system, the central nervous system, skin, appendix, GI tract (including but not limited to esophagus, duodenum, and colon), respiratory/pulmonary system (including but not limited to lung, nose, pharynx, larynx), eye, genito-reproductive system, gums, liver/biliary ductal system, renal system (including but not limited to kidneys, urinary tract, bladder), connective tissue (including but not limited to joints, cartilage), cardiovascular system, muscle, breast, lymphatic system, ear, endocrine/exocrine system (including but not limited to lacrimal glands, salivary glands, thyroid gland, pancreas), and bone/skeletal system. The immune response can be an inflammatory response associated with wound formation in any tissue, including but not limited to those mentioned herein.

Inflammatory diseases that affect the peripheral nervous system include, but are not limited to, radiculitis. Inflammatory diseases of the central nervous system include acute hemorrhagic leukoencephalitis, cholesterol granuloma, meningoencephalitis, optic neuritis, and Parsonage-Aldren-Turner syndrome, but are not limited to these diseases. Inflammatory diseases of the skin can include, but are not limited to, acute infantile hemorrhagic edema, contact dermatitis, Favre-Racouchot syndrome, folliculitis, panniculitis, Riehl's melanosis, Stevens-Johnson syndrome, and trichostasis spinulosa. Inflammatory diseases of the appendix include appendicitis.

Atrophic gastritis, Barrett's esophagus, Celiac disease, colitis, colonic diverticulitis, Curling's ulcers, Cushing's ulcers, esophagitis, phlegmonous gastritis, proctitis, toxic megacolon, and typhlitis are some inflammatory diseases that affect the GI tract. Inflammatory diseases of the respiratory/pulmonary system include, but are not limited to atrophic rhinitis, bronchiolitis obliterans organizing pneumonitis, pleural empyema, endogenous lipoid pneumonia, laryngeal granuloma, lymphocytic interstitial pneumonia, pharyngitis, pleuritis, sinusistis, and sterile pneumonitis. Inflammatory diseases of the eye can be blepharitis, dacryocystitis, endophthalmitis, Fuch's heterochromic cyclitis, giant papillary conjunctivitis, optic neuritis, phlyctenular keratoconjunctivitis, scleritis, but are not limited to these examples.

Diseases characterized by inflammation that affect the genito-reproductive system include, but are not limited to Bowenoid papulosis, cervicitis, cystitis, epidydymo-orchitis, peritonitis, and posthitis. Inflammatory diseases that affect the gums include cancrum oris, giant cell granuloma, gingivitis, pericoronitis, periodontitis, and pulpitis, but are not limited to these examples. Diseases states that are characterized by inflammation and that affect the liver/biliary ductal system include, but are not limited to, cholangitis and perihepatitis. Inflammatory diseases of the renal system can include chronic interstitial nephritis, Hunner's ulcer, post-streptococcal glomerulonephritis, and xanthogranulomatous pyelonephritis. Disease states that affect connective tissue include, but are not limited to, De Quervain's tenosynovitis, pyrophosphate arthropathy, reactive arthropathy, sacroilitis, synovitis, tenosynovitis, Tietze's costochondritis, and urate crystal arthropathy.

Disease states characterized by inflammation of the cardiovascular system include endocarditis, pericarditis, thrombophlebitis, and vasculitis, but are not limited to these examples. Inflammatory disease states that affect muscle include but are not limited to, myositis and Parsonage-Aldren-Turner syndrome. Mastitis and Mondor's disease of the breast are some inflammatory conditions that affect the breast. Diseases of the lymphatic system that are characterized by inflammation include mesenteric adenitis and pseudolymphoma, but are not limited to these examples. Inflammatory diseases of the ear can include diseases such as myringitis bullosa. Inflammatory diseases of the endocrine/exocrine system can include necrotizing sialometaplasia, pancreatitis, parotitis, and thyroiditis, while diseases of the bone/skeletal system characterized by inflammation include osteitis, osteitis fibrosa cystica, osteitis pubis, and periostitis, but are not limited to these examples. It is evident that many inflammatory diseases can be systemic and affect more than one organ system. Some systemic inflammatory diseases can include gangrene, Jarisch-Herxheimer reaction, and Reiter's syndrome.

Autoimmune disease is a class of diseases in which a subject's own antibodies react with host tissue or in which immune effector T cells are autoreactive to endogenous self-peptides and cause destruction of tissue. Autoimmune diseases include, but are not limited to, acquired factor VIII deficiency, acquired generalized lipodystrophy, alopecia greata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyly-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

Netrin Polypeptides and Analogs

As used herein with respect to proteins/peptides, the term “isolated” means separated from its native environment in sufficiently pure form so that it can be manipulated or used for any one of the purposes of the invention. Thus, isolated means sufficiently pure to be used (i) to raise and/or isolate antibodies, (ii) as a reagent in an assay, (iii) for sequencing, or (iv) in a therapeutic regimen, etc.

The term “or (a) fragment(s) thereof” as employed in the present invention and in context with polypeptides of the invention, comprises specific peptides, amino acid stretches of the polypeptides as disclosed herein. It is preferred that said “fragment(s) thereof” is/are functional fragment(s). The term “functional fragment” denotes a part of the above-identified polypeptide of the invention, which fulfills, at least in part, physiologically and/or structurally related activities of the polypeptide of the invention. As used herein, Netrin 1 functional activity comprises the ability to decrease inflammatory cell migration to a target site.

The polypeptide sequence of Netrin can be back translated to yield the corresponding nucleic acid, which can also be used in the methods of the present invention. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is readily manipulable by recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, as the term is used herein, because it is readily manipulable by standard techniques known to those of ordinary skill in the art.

Insertion of one or more pre-selected DNA sequences can be accomplished by homologous recombination or by viral integration into the host cell genome. The desired gene sequence can also be incorporated into the cell, particularly into its nucleus, using a plasmid expression vector and a nuclear localization sequence. Methods for directing polynucleotides to the nucleus have been described in the art. The genetic material can be introduced using promoters that will allow for the gene of interest, such as Netrin, to be positively or negatively induced using certain chemicals/drugs, to be eliminated following administration of a given drug/chemical, or can be tagged to allow induction by chemicals (including but not limited to the tamoxifen responsive mutated estrogen receptor) expression in specific cell compartments (including but not limited to the cell membrane).

Calcium phosphate transfection can be used to introduce plasmid DNA containing a target gene or polynucleotide such as Netrin into cells of interest and is a standard method of DNA transfer to those of skill in the art. DEAE-dextran transfection, which is also known to those of skill in the art, may be preferred over calcium phosphate transfection where transient transfection is desired, as it is often more efficient. Microinjection can also be particularly effective for transferring genetic material into the cells. This method is advantageous because it provides delivery of the desired genetic material directly to the nucleus, avoiding both cytoplasmic and lysosomal degradation of the injected polynucleotide. This technique has been used effectively to accomplish germline modification in transgenic animals. Cells of interest can also be genetically modified with nucleic acids expressing Netrin using electroporation.

Liposomal delivery of DNA or RNA to genetically modify cells of interest can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include LipofectinS (Life Technologies). Lipofectin, for example, is a mixture of the cationic lipid N-[1-(2,3-dioleyloyx)propyl]-N-N-N-trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA expressing Netrin into desired cells.

Naked plasmid DNA encoding Netrins of the present invention can be injected directly into a tissue mass. This technique has been shown to be effective in transferring plasmid DNA to skeletal muscle tissue, where expression in mouse skeletal muscle has been observed for more than 19 months following a single intramuscular in ection. More rapidly dividing cells take up naked plasmid DNA more efficiently. Therefore, it is advantageous to stimulate cell division prior to treatment with plasmid DNA. Microprojectile gene transfer can also be used to transfer genes into stem cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors can be used to deliver nucleic acids encoding Netrin 1 to cells and/or tissues of interest. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors that can be used to deliver nucleic acids encoding Netrins of the present invention include, but are not limited to, adenoviral vectors, adeno-associated viral vectors, retroviral vectors (including lentiviral vectors), alphaviral vectors (e.g., Sindbis vectors), and herpes virus vectors.

Non-protein Netrin analogs having a chemical structure designed to mimic Netrin 1 functional activity can also be administered according to methods of the invention. Netrin analogs may exceed the physiological activity of native Netrins (e.g., Netrin 1). Methods of analog design are well known in the art, and synthesis of analogs can be carried out according to such methods by modifying the chemical structures such that the resultant analogs exhibit enhance selectivity to the binding grove of a Netrin receptor and thus are able to successfully compete with the native Netrin for the receptor binding site(s). These chemical modifications include, but are not limited to, substituting alternative R groups and varying the degree of saturation at specific carbon atoms of the native Netrin molecule. Preferably, the Netrin analogs are relatively resistant to in vivo degradation, resulting in a more prolonged therapeutic effect upon administration. Assays for measuring functional activity include, but are not limited to, those described in the Examples below.

Antibodies that bind to a Netrin receptor (e.g., UNC5H2), can also mimic Netrin 1 functional activity and can be administered according to methods of the invention. Such antibodies may exceed the physiological activity of native Netrins.

Antibodies are well known to those of ordinary skill in the science of immunology. As used herein, the term “antibody” means not only intact antibody molecules but also fragments of antibody molecules retaining immunogen binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv) and fusion polypeptides. Preferably, the antibodies of the invention are monoclonal. Alternatively the antibody may be a polyclonal antibody. The preparation and use of polyclonal antibodies is also known to one of ordinary skill in the art. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing UNC5H2, or immunogenic fragments thereof, as an immunogen. One method of obtaining antibodies is to immunize suitable host animals with an immunogen and to follow standard procedures for polyclonal or monoclonal production. The immunogen will facilitate presentation of the immunogen on the cell surface. Immunization of a suitable host can be carried out in a number of ways. Nucleic acid sequences encoding UNC5H2, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding UNC5H2, or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205. In one another version, the heavy chain and light chain C regions are replaced with human sequence. In another version, the CDR regions comprise amino acid sequences for recognition of antigen of interest, while the variable framework regions have also been converted to human sequences. See, for example, EP 0329400. It is well established that non-CDR regions of a mammalian antibody may be replaced with corresponding regions of non-specific or hetero-specific antibodies while retaining the epitope specificity of the original antibody. This technique is useful for the development and use of humanized antibodies in which non-human CDRs are covalently joined to human FR and/or Fc/pFc′ regions to produce a functional antibody. In a third version, variable regions are humanized by designing consensus sequences of human and mouse variable regions, and converting residues outside the CDRs that are different between the consensus sequences.

Construction of phage display libraries for expression of antibodies, particularly the Fab or scFv portion of antibodies, is well known in the art (Heitner, 2001). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected immunogen, thereby isolating one or more species of phagemid containing particular heavy and light chain-encoding genes and antibody molecules that immunoreact with the preselected immunogen. The preselected immunogen can be provided by or obtained from cells of the invention that express UNC5H2, or immunogenic fragments thereof, on the cell surface.

Single chain variable region fragments are made by linking light and heavy chain variable regions by using a short linking peptide. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or a portion thereof. Compositions comprising a biphasic scFv could be constructed in which one component is a polypeptide that recognizes an immunogen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector for the production of scFvs is pcDNA3 (Invitrogen, San Diego, Calif.) in mammalian cells, described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures, may be necessary or useful to optimize production of properly folded scFv in prokaryotic systems; or, it may be preferable to express scFv in eukaryotic cells.

Pharmaceutical Compositions

The present invention contemplates pharmaceutical preparations comprising Netrin polypeptide molecules or other functional substitutes, such as Netrin analogs or UNC5H2 antibodies, together with pharmaceutically acceptable carriers. Polypeptides of the invention may be administered as part of a pharmaceutical composition. The compositions should be sterile and contain a therapeutically effective amount of the polypeptides in a unit of weight or volume suitable for administration to a subject.

Pharmaceutical compositions of the invention to be used for therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous Netrin polypeptide solution, such as an aqueous solution of Netrin polypeptides, and the resulting mixture can then be lyophilized. The infusion solution can be prepared by reconstituting the lyophilized material using sterile Water-for-Injection (WFI).

The polypeptides, analogs or antibodies (i.e., active agents) may be combined, optionally, with a pharmaceutically acceptable carrier. The term “pharmaceutically-acceptable carrier” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

Netrin polypeptides of the present invention can be contained in a pharmaceutically acceptable carrier. The carrier preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result. In some cases this is a local (site-specific) reduction of inflammation. In other cases, it is inhibition of systemic infection and/or sepsis. Generally, doses of active polypeptide compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the Netrin polypeptide compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising Netrin polypeptides can be added to a physiological fluid such as blood or synovial fluid. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g, tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising Netrin polypeptides of the present invention can contain multivalent metal ions, such as calcium ions, magnesium ions and/or manganese ions. Any multivalent metal ion that helps stabilize the Netrin polypeptide composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the Netrin polypeptide(s) contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of Netrin polypeptides, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and Netrin polypeptides are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the Netrin compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Compositions and methods of the invention can be used in combination with existing anti-inflammatory treatment modalities, including but not limited to, drug therapy, and administration with anti-inflammatory cytokines. Methods of the invention can optionally comprise contacting inflammatory cells with Netrin polypeptides in combination with other anti-inflammatory drug treatments such as, but not limited to, antihistamines, non-steroidal anti-inflammatory agents (NSAIDs), eicosanoid receptor antagonists, cytokine antagonists, monoclonal antibodies, 3-hydroxymethylglutaryl-coenzyme A (HMG-CoA) reductase inhibitors, and corticosteroids (see, for example, Goodman and Gilman's The Pharmacological Basis of Therapeutics).

Antihistamines fall generally under three broad classes, according to the histamine receptor subtype they antagonize and display specificity for. Histamine H1 receptors are primarily responsible for the anti-inflammatory response, while H2 receptors are limited to gastric acid secretion. Histamine H1 receptor antagonists include, but are not limited to, carbinoxamine, clemastine, diphenhydramine, dimenhydrinate, pyrilamine, tripelennamine, chlorpheniramine, brompheniramine, chlorcyclizine, acrivastine, promethazine, as well as piperazines such as astemizole, levocabastine, hydroxyzine, cyclizine, cetirizine, meclizine, loratadine, fexofenadine, and terfenadine.

NSAIDs include the salicylate derivatives, para-aminophenol derivatives, indole and indene acetic acids, heteroaryl acetic acids, arylpropionic acids, anthranilic acids (also known in the art as fenamates), enolic acids, and alkanones. Salicylate derivates include aspirin, sodium salicylate, choline magnesium trisalicylate, salsalate, diflunisal, salicylsalicylic acid, sulfasalazine, and olsalazine, but are not limited to these drugs. Para-aminophenol derivates are exemplified by acetaminophen. Indomethacin, sulindac, and etodolac comprise indole and indene acetic acids, while heteroaryl acetic acids include tolmetin, diclofenac, and ketorolac. Examples of arylpropionic acids include ibuprofen, naproxen, flurbiprofen, ketoprofen, fenoprofen, and oxaprozin. Fenamates include but are not limited to mefenamic acid and meclofenamic acid. Some examples of enolic acids include the oxicams piroxicm and tenoxicam, and pyrazolidinediones such as phenylbutazone and oxyphenthatrazone. Alkanones can comprise nabumetone.

Eicosanoid receptor antagonists include, but are not limited to, leukotriene modifiers, which can act as leukotriene receptor antagonists by selectively competing for LTD-4 and LTE-4 receptors. These compounds include, but are not limited to, zafirlukast tablets, zileuton tablets, and montelukast. Zileuton tablets function as 5-lipoxygenase inhibitors. Cytokine antagonists can comprise anti-TNFα antibodies, and fusion proteins of the ligand binding domain of the TNFα receptor and the Fc portion of human immunoglobulin G1. Other cytokine antagonists include recombinant human interleukin-1 receptor antagonist, recombinant human IFNα, recombinant human IFNβ, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, downregulators of IgE, among others.

Monoclonal antibodies can advantageously impede leukocyte rolling and binding of extracellular matrix proteins, glycoproteins, and carbohydrates. Such antibodies have been directed against the mucins sialyl Lewis^{X}, the integrins, the E, P, and L-selectins, and other adhesion molecules. Other potential targets for monoclonal antibodies include cytokine receptors such as TNFαR, the interleukin receptors, interferon receptors, among others.

HMG-CoA reductase inhibitors (or statins) are drugs used to lower cholesterol by impinging on a key enzyme in the cholesterol biosynthetic pathway, 3-hydroxy-3-methylglutaryl-coenzyme A reductase (HMG-CoA reductase) (reviewed in Weitz-Schmidt, G; Trends Pharm. Sci. 23(10): 482-6.). These drugs are collectively known as statins, which also impinge on leukocyte migration (Diomede, L. et al. Arterioscler. Thromb. Vasc. Biol. 21(8): 1327-32.). Downregulation of the cytokines MCP-1, IL-6, and the chemokine RANTES has been observed, as well as downregulation of endothelial and leukocyte adhesion molecules (Yoshida, M. et al., Arterioscler. Thromb. Vasc. Biol. 21(7): 1165-71; Romano, M. et al. Lab. Invest. 80(7): 1095-1100). Additionally, statins may cause smooth muscle relaxation, and downregulation of cytokine and chemokine release (Niwa, S. et al. Int. J. Immunopharmacol. 18(11): 669-75). Examples of statins include, but are not limited to, mevastatin, lovastatin, simvastatin, pravastatin, and fluvastatin.

Corticosteroids cause a decrease in the number of circulating lymphocytes as a result of steroid-induced lysis of lymphocytes, or by alterations in lymphocyte circulation patterns (Kuby, J. (1998) In: Immunology 3^{rd} Edition W.H. Freeman and Company, New York; Pelaia, G. et al. Life Sci. 72(14): 1549-61). Corticosteroids affect the regulation of nuclear factor κB (NF-κB) by inducing the upregulation of an inhibitor of NF-κB known as IκB, which sequesters NF-κB in the cytoplasm and prevents it from transactivating pro-inflammatory genes in the nucleus. Corticosteroids also reduce the phagocytic ability of macrophages and neutrophils, as well as reducing chemotaxis. Examples of corticosteroids are alclometasone, amcinonide, beclomethasone, betamethasone, clobetasol, clocortolone, cortisol, hydrocortisone, prednisolone, and prednisone, but are not limited to these examples.

Methods of the invention can optionally comprise contacting inflammatory cells with Netrin polypeptides in combination with other anti-inflammatory cytokines such as, but not limited to, interleukin-4 (IL-4), interleukin-10 (IL-10), interleukin-13 (IL-13), interleukin-16 (IL-16), interleukin-1 receptor antagonist (IL-1ra), interferon α (IFNα), transforming growth factor-β (TGF-β), among others. The cytokines may be administered together or separately in combination with Netrin polypeptides in the compositions and methods described herein.

The balance between pro-inflammatory cytokines and anti-inflammatory cytokines determines the net effect of an inflammatory response. The type, duration, and also the extent of cellular activities induced by one particular cytokine can be influenced considerably by the nature of the target cells, the micro-environment of a cell, depending, for example, on the growth and activation state of the cells, the type of neighboring cells, cytokine concentrations, the presence of other cytokines, and even on the temporal sequence of several cytokines acting on the same cell.

Screening

Methods of the invention additionally comprise methods for the identification and selection of Netrin analogs and/or functional equivalents, referred to herein as “screening methods.”

Netrins of the invention function through receptor binding (e.g., binding to UNC5H2). Accordingly, test compounds can be assayed, or “screened” to identify those compounds that have Netrin functional activity.

The ability of the test compound to bind to a Netrin receptor such as UNC5H2 can be evaluated according to methods known in the art. This can be accomplished, for example, by coupling the compound, e.g., the substrate, with a radioisotope or enzymatic label such that binding of the compound, e.g., the substrate, to receptors can be determined by detecting the labeled compound, e.g., substrate, in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a Netrin receptor or a biologically active portion thereof is contacted with a test compound and the ability of the test compound to bind to the channel or biologically active portion thereof is evaluated. Preferably, the cell-free assay comprises a membrane. Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a Netrin receptor can be accomplished using real-time Biomolecular Interaction Analysis (BIA) (see, e.g., Sjolander, S. and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the sample comprising the Netrin receptor or the test compound is anchored onto a solid phase. The channel/test compound complexes anchored on the solid phase can be detected at the end of the reaction.

It may be desirable to immobilize either the Netrin receptor, an anti-Netrin receptor antibody or its target molecule to facilitate separation of complexed from uncomplexed forms of one or both of the proteins, as well as to accommodate automation of the assay. Binding of a test compound to a Netrin receptor, or interaction of a Netrin receptor with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/Netrin receptor fusion proteins can be adsorbed onto glutathione sepharose beads (Sigma Chemical, St. Louis, Mo.) or glutathione derivatized microtiter plates, which are then combined with the test compound or the test compound and a sample comprising the GST-tagged Netrin receptor, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Other techniques for immobilizing a complex of Netrin receptors on matrices include using conjugation of biotin and streptavidin. For example, biotinylated proteins can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with an epitope on the Netrin receptor but which do not interfere with binding of the Netrin receptor to a test compound. Such antibodies can be derivatized to the wells of the plate, and unbound target or Na⁺ channels trapped in the wells by antibody conjugation. Methods for detecting such complexes, in addition to those described above for the GST-immobilized complexes, include immunodetection of complexes using antibodies reactive with a component of the Netrin receptor, as well as enzyme-linked assays which rely on detecting an enzymatic activity associated with the channel.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the Netrin receptor, e.g., by including a membrane component or synthetic membrane components.

In a specific embodiment, the assay includes contacting the Netrin receptor or biologically active portion thereof with a known compound which binds the receptor to form an assay mixture, contacting the assay mixture with a test compound, and determining the ability of the test compound to interact with a Netrin receptor, wherein determining the ability of the test compound to interact with a Netrin receptor includes determining the ability of the test compound to preferentially bind to the Netrin receptor, or to modulate the activity of the Netrin receptor, as compared to the known compound.

The Netrin receptors described herein (e.g., UNC5H2) can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules.

To identify compounds that interfere with the interaction between the Netrin receptors and an extracellular binding partner(s), a reaction mixture containing the target gene product and the binding partner is prepared, under conditions and for a time sufficient, to allow the two products to form a complex. In order to test an inhibitory agent, the reaction mixture is provided in the presence and absence of the test compound. The test compound can be initially included in the reaction mixture, or can be added at a time subsequent to the addition of the Netrin receptor and its cellular or extracellular binding partner. Control reaction mixtures are incubated without the test compound or with a placebo. The formation of any complexes between the Netrin receptor and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and a Netrin receptor comprising one or more mutant amino acids. This comparison can be important in those cases wherein it is desirable to identify compounds that disrupt interactions of mutant but not normal Netrin receptors.

These assays can be conducted in a heterogeneous or homogeneous format. Heterogeneous assays involve anchoring either the Netrin receptor or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the Netrin receptors or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the Netrin receptor proteins or fragments thereof can be used as “bait proteins” in a two-hybrid assay or three-hybrid assay (see, e.g., U.S. Pat. No. 5,283,317; Zervos et al., Cell 72:223-232, 1993; Madura et al., J. Biol. Chem. 268:12046-12054, 1993; Bartel et al., Biotechniques 14:920-924, 1993; Iwabuchi et al., Oncogene 8:1693-1696, 1993; and Brent WO94/10300), to identify other proteins, which bind to or interact with Netrin receptor proteins (“Netrin receptor-binding proteins”) and are involved in Netrin receptor activity. Such Netrin receptors can be activators or inhibitors of signals by the Netrin receptor or Netrin receptor-sensitive targets.

The two-hybrid system is based on the modular nature of most transcription factors, which consist of separable DNA-binding and activation domains. Briefly, the assay utilizes two different DNA constructs. In one construct, the gene that codes for a Netrin receptor or fragment thereof (e.g., corresponding to a soluble portion of an extracellular domain of the receptor) is fused to a gene encoding the DNA binding domain of a known transcription factor (e.g., GAL-4). In the other construct, a DNA sequence, from a library of DNA sequences, which encodes an unidentified protein (“prey” or “sample”) is fused to a gene that codes for the activation domain of the known transcription factor. (Alternatively, the Netrin receptor can be the fused to the activator domain.) If the “bait” and the “prey” proteins are able to interact, in vivo, forming a Netrin receptor-dependent complex, the DNA-binding and activation domains of the transcription factor are brought into close proximity. This proximity allows transcription of a reporter gene (e.g., lacZ) that is operably linked to a transcriptional regulatory site responsive to the transcription factor. Expression of the reporter gene can be detected and cell colonies containing the functional transcription factor can be isolated and used to obtain the cloned gene that encodes the protein that interacts with the Netrin receptor.

In another aspect, the invention pertains to a combination of two or more of the assays described herein. For example, a Netrin analog or functional equivalent can be identified using a cell-based or a cell free assay, and the ability of the agent to modulate the activity of a Netrin receptor can be confirmed in vivo, e.g., in an animal such as an animal model for a pain disorder or a disorder associated with stroke or traumatic brain injury.

This invention further pertains to novel agents identified by the above-described screening assays. Accordingly, it is within the scope of this invention to further use an agent identified as described herein in an appropriate animal model to determine the efficacy, toxicity, side effects, or mechanism of action, of treatment with such an agent. Furthermore, novel agents identified by the above-described screening assays can be used for treatments as described herein.

The test compounds of the present invention can be obtained singly or using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

Chemical compounds to be used as test compounds (i.e., potential receptor agonists) can be obtained from commercial sources or can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In one aspect the compounds are organic small molecules, that is, compounds having molecular weight less than 1,000 amu, alternatively between 350-750 amu. In other aspects, the compounds are: (i) those that are non-peptidic; (ii) those having between 1 and 5, inclusive, heterocyclyl, or heteroaryl ring groups, which may bear further substituents; (iii) those in their respective pharmaceutically acceptable salt forms; or (iv) those that are peptidic.

The term “heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring can be substituted by a substituent.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring can be substituted by a substituent.

The term “substituents” refers to a group “substituted” on an alkyl, cycloalkyl, aryl, heterocyclyl, or heteroaryl group at any atom of that group. Suitable substituents include, without limitation, alkyl, alkenyl, alkynyl, alkoxy, halo, hydroxy, cyano, nitro, amino, SO₃H, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

Combinations of substituents and variables in compounds envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., transport, storage, assaying, therapeutic administration to a subject).

Methods of the invention will additionally involve screening compositions for Netrin functional activity. Methods well known to the skilled artisan can be used to detect changes in inflammatory cell phenotypes in response to Netrin polypeptides of the present invention. The detection methods disclosed herein can be used with the compositions and methods of the present invention to modulate immune cells in vivo or ex vivo, i.e., these detection methods can be carried out in patients to monitor therapy. They can also be used in methods of screening compositions for Netrin functional activity in vitro.

For example, the methods can include comparing the value or the profile (i.e., multiple values) of an inflammatory cell modulated by a Netrin polypeptide or analog to a reference value or reference profile of a control. One such value or profile is a gene expression profile. The gene expression profile of an inflammatory cell modulated by a Netrin polypeptide or analog can be obtained by any of the methods described herein (e.g., by assaying expression profile following activation of the Netrin receptor).

Values and/or profiles can be detected and compared by procedures well known in the art such as microarray analysis, calorimetric assays such as the Bradford Assay and Lowry Assay, RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA.

Changes in tissue or organ morphlogy as a result of inflammation further comprise values and/or profiles that can be assayed by methods of the invention by any method known in the art, including x-ray, sonogram and ultrasound.

Modulation of inflammatory cells can be also be detected by measuring the changes in levels of adhesion molecules at the nucleic acid and/or protein levels. Adhesion molecules to be assayed include, but are not limited to, the integrins LFA-1 (also known in the art as CD11a/CD18, or α_{L}β₂), Mac-1 (CD11b/CD18, MO-1, CR3, αₘβ₂), gp150/95 (CD11c/CD18, αₓβ₂), VLA-4 (α₄β₁), and α_{IIb}β₃. The ligands or otherwise known as “counter-receptors” associated with the above integrins include, but are not limited to, ICAM-1, ICAM-2, ICAM-3, fibrinogen, C3b 1, Factor X, VCAM-1, fibronectin, vitronectin, thrombospondin, and von Willebrand factor (reviewed in Gahmberg, C. G., et al., (1998) Cell. Mol. Life Sci. 54: 549-555). Selectins comprise E-selectin (CD62E, ELAM-1, LECAM-1); P-selectin (CD62P; PADGEM, GMP140); and L-selectin (MAdCAM-1, LAM-1, MEL-14, Leu-89). The ligands and/or counter-receptors associated with the above selectins include sialylated fucosylated lactosamines (e.g., sialyl Lewis^{X}, sialyl Lewis^{A}), L-selectin, PSGL-1, ESL-1, Sgp50 (GlyCAM-1), Sgp90 (CD34). ICAM family members include, but are not limited to, LFA-2 (CD2); LFA-3 (CD58); ICAM-1 (CD54), ICAM-2 (CD102), ICAM-3 (CD50), VCAM-1 (CD106), PECAM-1 (CD31) (reviewed in Crockett-Torabi, E., J. Leuk. Biol. 63: 1-14).

The majority of receptors for chemotactic agents are the seven transmembrane G-protein coupled receptors, which are well characterized in the art (see, for example, Ono, S. J., et al., J. Allergy Clin. Immunol. 111: 1185-99). Netrin receptors of the invention can be G-protein coupled receptors. The released α- and βγ-subunits of G-protein coupled receptors result in activation of phospholipase subtypes C, β, and D. The action of PLC-β on the lipid phosphatidylinositol-4,5-bisphosphate (PIP₂) causes cleavage to form inositol-1,4,5-trisphosphate, or IP₃, which causes release of intracellular calcium from central and peripheral organelle stores. In addition, phosphatidylinositol-3-kinase (PI3K) is activated by G-proteins and can phosphorylate PIP₂ to PIP₃ and other lipid second messengers. Inflammatory cells contain these calcium stores, and chemotactic agents cause Ca²⁺ release. Thus, PI3K activation can be used as a value or profile monitored to assess Netrin functional activity.

Intracellular calcium release can be detected by microscopy or alternatively, fluorescence emissions, in isolated cells using calcium-binding dyes exemplified by Fura analogs, Indo-1, Calcium Green, Calcium Orange, Fluo-3, and Fluo-4. Other methods that could be used to detect changes in calcium flux include electrophysiology, and detection of calcium binding to the calcium-sensitive photoproteins, aqueorin and obelin.

Change in levels of the nucleotide second messenger cyclic adenosine monophosphate (cAMP) is also a measurable parameter to monitor in assessing Netrin functional activity. cAMP levels drop in response to activation of chemokine receptors through G_{iα}. Without wanting to be bound by theory, it is believed that the inhibition of adenylyl cyclase is mediated by the α subunit of the G_{iα} receptor, while the βγ subunits may be involved in the activation of PLC, subsequently releasing calcium stores through IP₃. cAMP levels can be directly measured through commercially available kits, with or without radionucleotides. A frequently used assay is an enzyme immunoassay, using a cAMP-binding antibody coupled to chemiluminescent or fluorescent probes. Another method that can be advantageously used to detect changes in cAMP levels is Scintillation Proximity Assay (SPA) technology, which involves immobilizing the receptor onto a bead. When a suitably radiolabeled ligand, such as radiolabeled cAMP, binds to the immobilized receptor, the radioligand will be in close proximity to the bead and will stimulate the bead to emit light. SPA technology eliminates the need to separate antibody bound from free ligand common to heterogeneous radioimmunoassays, which can be difficult to detect in low affinity binding events where a separation step might shift or even disrupt the equilibrium of the binding. Association and dissociation of a radiolabeled ligand to and from the receptor can be monitored in real time.

Cell migration causes gross, well-documented changes in inflammatory cell morphology that can also be monitored to assess Netrin functional activity. The cell in its “resting” state is spherical, however within seconds of the arrival of a chemotactic stimulus, membrane ruffling occurs over the whole surface of the cell. Several membrane ruffles can become larger, forming filopodia and lamellipodia (reviewed in Pettit, E. J. and Fay, F. S., Physiol. Rev. 78: 949-967). These morphological changes are especially dramatic when the binary signal of integrin engagement plus TNF-, chemokine-, or complement-mediated stimulation triggers massive degranulation of the inflammatory cell, and a phenomenon known as “respiratory burst” (Nathan, C. F. J. Clin. Invest. 80(6): 1550-60.). The components of this respiratory burst include proteases, hydrolases, bacterial permeability increasing factor, α-defensins, serprocidins, azurocidin, and factors that promote formation of reactive oxygen species (ROS), like hydrogen peroxide, hypohalites, and chloramines.

Changes in cell shape and morphology can be detected by light microscopy and immunocytochemistry. Particularly useful is the filamentous actin-binding dye rhodamine, which can be easily detected by fluorescence microscopy and can detect minute changes in the actin cytoskeleton in response to chemotactic stimuli (i.e., stress fibers). Another structural protein that undergoes changes in its conformation is microtubules, which can be detected using immuncytochemistry. Other target proteins that can be used as markers for cell morphology and shape changes are actin binding proteins α-actinin, calpactin, fimbrin, filamin, myosin, but are not limited to these examples.

Formation of reactive oxygen species (ROS) and reactive nitrogen species (RNS) occurs during “respiratory burst”, or degranulation of inflammatory cells such as neutrophils. Oxygen consumption in inflammatory cells is increased through the activity of an NADPH-oxidase that generates superoxide anion and hydrogen peroxide. These oxygen metabolites give rise to yet other reactive oxygen species that are strongly anti-microbial but can also cause collateral damage to surrounding tissues. Several dyes have been developed that after being excited by reactive oxygen species, release chemiluminescent energy (Dahlgren, C., and Karlsson, A., J. Immunol. Meth. 232: 3-14). Luminol and isoluminol are two such dyes. Luminol is an activity amplifier, and the sensitivity is very high due to the high quantum yield of the molecule. A plurality of commercially available dyes can be used as well, and include 10-acetyl-3,7-dihydroxy-2-dodecylphenoxazine, 6-amino-6-deoxyluciferin, aminophenylfluorescein, 5-carboxy-2′,7′-dichlorodihydrofluorescein diacetate, coumarin-3-carboxylic acid, 2′,7′-dichlorodihydrofluorescein diacetate, dihydrocalcein, dihydroethidium, dihydrorhodamine 123, dihydrorhodamine 6G, glutathione ethyl ester, hydroxyphenylfluorescein, trans-1-(2′-methoxyvinyl)pyrene, cis-parinaric acid, 5-(pentafluorobenzoylamino)dihydrofluorescein diacetate, RedoxSensor™, among others. Production of hydrogen peroxide can be detected by oxidation of p-hydroxyphenylacetate molecules in the presence of horseradish peroxidase. Fluorescence emission at 405 nm can be detected when activated at 317 nm.

The practice of the present invention employs, unless otherwise indicated, conventional techniques of molecular biology (including recombinant techniques), microbiology, cell biology, biochemistry and immunology, which are well within the puview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention.

Also contemplated by the present invention are databases containing data generated from the methods of the instant invention. For example, a database can be provided, which holds all of the data generated for a particular cell or disease, the screening of a complete or incomplete compound library, data from a screening method for Netrin functional activity in accordance with the instant invention, or any other type of data generated from the methods of the instant invention. The invention further contemplates providing access to the database for commercial purposes. Access can be electronic access over a global communications network, such as the World Wide Web.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### UNC5H2 Interacts with G-Proteins

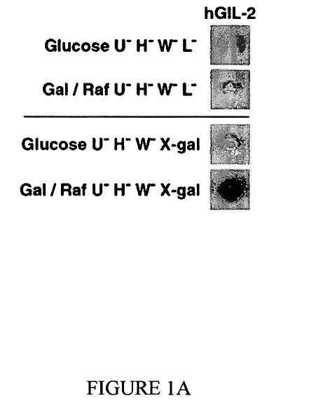

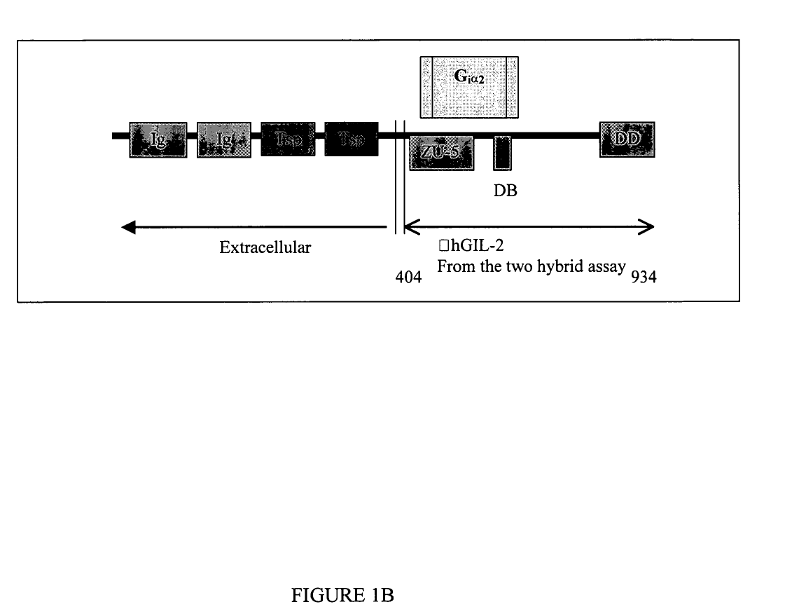

The yeast two-hybrid system was used to identify proteins that interact with GTPase deficient G_{iα2}, gip2, potentially regulating its signaling pathway. This G-protein is central to chemokine receptor-induced chemotaxis and a potential target of the UNC5H2/Netrin pathway (Spangrude, G. J., et al., J. Immunol. 132: 354-62; Chaffin, K. E., et al., Eur. J. Immunol. 21: 2565-73). Using the yeast two-hybrid assay to screen a chimeric human lung cDNA library (FIG. 1A), five molecules that interacted with gip2 were identified: eIF3-p48 (found as a false positive in many screens), EPC-1 (a secreted factor), human nicotinamide N-methyltransferase (a mitochondrial enzyme), RACK1 and UNC5H2. A yeast clone expressing LexA-gip2 and B42-hGIL-2 showed growth on galactose (Gal) media lacking uracil, histidine, tryptophan and leucine (Gal U- H- W- L-). Blue precipitate on X-gal indicates cumulative β-galactosidase activity, resulting from activation of the lacZ reporter gene by gip² and hGIL-2 interaction. A clone, hGIL-2, was identified that contained the 531-carboxyl amino acid of UNC5H2 (FIG. 1B) (Komtasuzaki, K., et al., Biochem. Biophys. Res. Commun. 297: 898-905). BLAST analysis (NIH) of this clone revealed 92% identity with rat UNC5H2 (GenBank NP_{—}071543) at the amino acid level. A similar identity was seen for mouse UNC5H2. Other members of the UNC5H family were less similar (54% to human UNC5H1 and 63% to human UNC5C) and it was concluded that this clone was indeed human UNC5H2. Notably, the interaction of gip2 and UNC5H2 occurred via the cytoplasmic tail of the human UNC5H2 (FIG. 1B). FIG. 1B also depicts a graphic representation of clone hGIL-2 and human UNC5H2 protein, and its various predicted protein domains: Ig: immunoglobulin domain; TM: transmembrane domain; ZU-5: domain present in ZO-1 and UNC5; DB: DCC binding domain; DD: death domain.

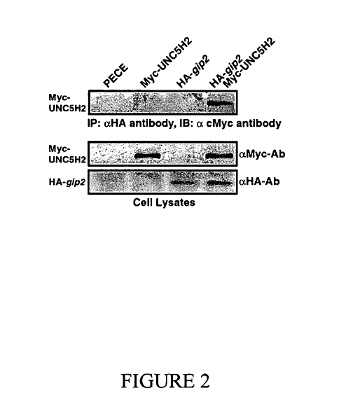

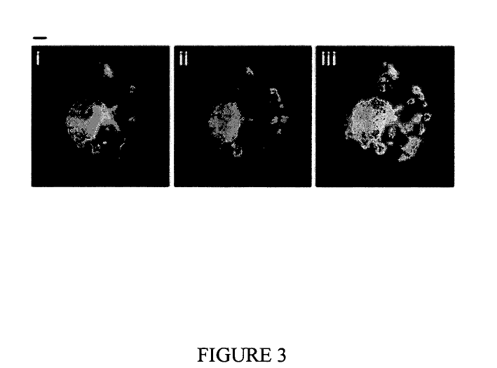

To determine whether UNC5H2 associates with G_{iα2} in vitro, COS7 cells were transfected with pHA-gip2 and pMyc-UNC5H2. Hemagglutinin-tagged gip2 was precipitated from lysates with a rabbit polyclonal antibody to the hemagglutinin epitope (Clontech) and immunoblotted with anti-Myc monoclonal antibody (FIG. 2). Myc tagged UNC5H2 co-immunoprecipitated with HA-gip2. These findings indicate that constitutively active G_{iα2} and UNC5H2 associate in vitro. To further confirm this interaction, deletions of UNC5H2 were constructed. Only constructs containing the cytoplasmic domain of UNC5H2 co-immunoprecipitated with HA-gip2, confirming that G_{iα2} interacts with the cytoplasmic domain of UNC5H2. To determine that this interaction occurred in vivo, immunofluorescence studies were performed in COS7 cells that were transfected with pHA-G_{iα2} and pMyc-UNC5H2. G_{iα2} and UNC5H2 colocalized in both the plasma membrane and the cytoplasm (FIG. 3).

### Example 2

### UNC5H2 Interacts with GTP-Activated form of G_{iα2} G-Protein

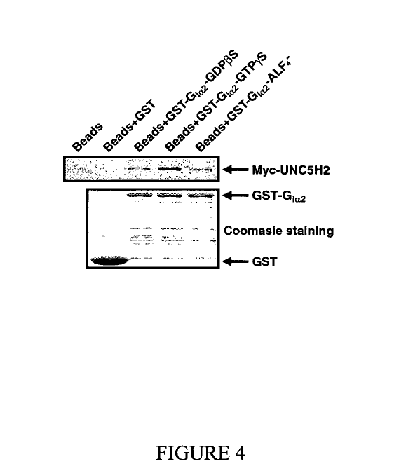

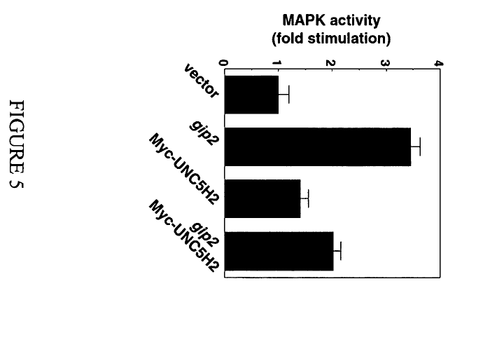

G-proteins can cycle between 3 states (GDP- or GTP-bound and a transitional state) and are likely to interact with different molecules during each state. GST-tagged G_{iα2} was tested for the ability to bind UNC5H2 from COS7 cells induced to over-express Myc-UNC5H2. Studies were performed with G_{iα2} bound to GDP, or GTP, or in the transitional state. G_{iα2} in the GTP-bound state showed the greatest binding to UNC5H2, indicating a state-dependent preference for this interaction (FIG. 4). This is consistent with previous findings, where constitutively activated G_{iα2} bound UNC5H2. Taken together, these data indicate that UNC5H2 interacts with the GTP charged activated form of G_{iα2}. cAMP and MAPK are important intermediates in the Netrin 1 pathway. These pathways are also regulated by G_{iα2}. Whether UNC5H2 could affect G_{iα2}'s regulation of these signaling pathways was investigated. To address whether G_{iα2} activates mitogen-activated protein kinases (MAPK) independently of the βγ subunit (Pace, A. M., et al., Mol. Biol. Cell 6:1685-95), Rat-1a cells were co-transfected with p44MAPK, and either pMyc-UNC5H2, pHA-gip2 (constitutively-activated G_{iα2}), pMyc-UNC5H2 plus pHA-gip2 or empty plasmid controls. A kinase assay was performed on lysates from these transfected cells (Kinane, T. B., et al, Am. J. Physiol. 272: F273-82). MAPK activity was upregulated 3.5-fold by gip2. However, co-expression of pMyc-UNC5H2 and gip2 decreased MAPK activation almost to baseline levels (FIG. 5). In addition, βγ subunit signaling was investigated and this signaling pathway was not altered (not shown). Thus, these experiments demonstrate that UNC5H2 inhibits G_{iα2} induced signaling via MAPK.

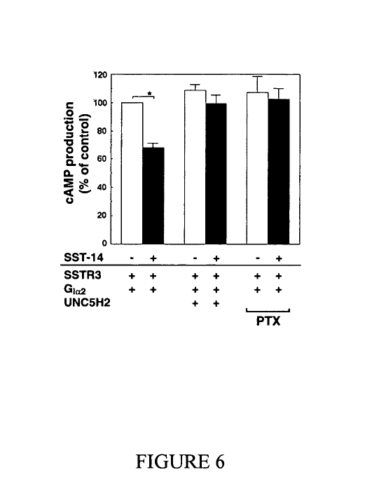

Inhibition of adenylyl cyclase, with a corresponding drop in intracellular cAMP levels, is a well-established effect of G_{iα2} activation. It has been shown that a deficiency of G_{iα2} increases intracellular cAMP levels, whereas expression of gip2 leads to chronic suppression of cAMP levels (Moxham, C. M. et al., Nature 379: 840-4). UNC5H2 and G_{iα2} may interact to regulate cAMP levels in the cell. To explore this potential regulation, the responses in the context of a G-protein coupled receptor were examined. The somatostatin receptor 3 (SSTR3) is coupled to G_{iα2} and G_{iα1}, and in the presence of ligand represses adenylyl cyclase (Komatsuzaki, K., et al., FEBS Lett. 406: 165-70). This was a useful receptor pathway to test the functional consequences of the G_{iα2}-UNC5H2 interaction, as it was reconstituted in COS7 cells, which do not express G_{iα1}. COS7 cells were co-transfected with pCMV6c-SSTR3 and pcDNAI-G_{iα2}, as well as pMyc-UNC5H2 or empty vectors. Cells were treated with somatostatin-14 (SST-14) (Bachern) or vehicle for 30 minutes. As expected, SST14 reduced cAMP accumulation by 32% (FIG. 6) (Komatsuzaki, K., et al., FEBS Lett. 406-165-70). This repression was abolished by either co-transfection with UNC5H2 or by treatment with the by Gᵢ inhibitor, pertussis toxin (PTX). This suggested that UNC5H2 abolished Gi_{α2}'s effects. Thus, Netrin 1, the natural ligand of UNC5H2, could alter this interaction by either releasing G_{iα2} or by further enhancement of G_{iα2} binding.

Netrin 1 treatment converted the SSTR3 receptor into an enhancer of cAMP accumulation, increasing cAMP by 41% (R&D Systems 11 g/ml for 15 minutes prior to SST-14 treatment). Thus G_{iα2}'s effects on cAMP were abolished and SSTR3 functioned as if it was coupled to G_{αs}. Together, the data demonstrate that UNC5H2 abolishes G_{iα2} signaling. In summary, upon binding of Netrin, UNC5H2 binds the activated G-protein, preventing it from signaling. The βγ subunit, however, is still active and activates β adrenergic receptor kinases, which downregulate the GPCR's.

### Example 3

### Extra-CNS Expression of the UNC5H2 Netrin Receptor

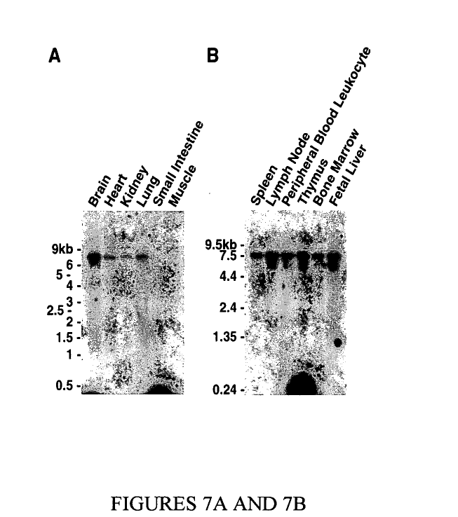

Netrin displays extra-CNS expression, and it is predicted that its receptor, UNC5H2, is also expressed outside of the brain (Leonardo, et al., Nature 386: 833-8). Additionally, a related molecule, UNC5H3, is expressed in the developing lung, kidney and cartilage (Przyborski, et al., Development (Supplement) 125: 41-50). Northern blot analysis demonstrated that UNC5H2 is strongly expressed in the lung, brain, and to a lesser degree in heart and kidney (FIG. 7A). This receptor is also strongly expressed in hematopoietic and immune tissues, including spleen, lymph node, peripheral leukocytes, thymus, bone marrow and the fetal liver (FIG. 7B).

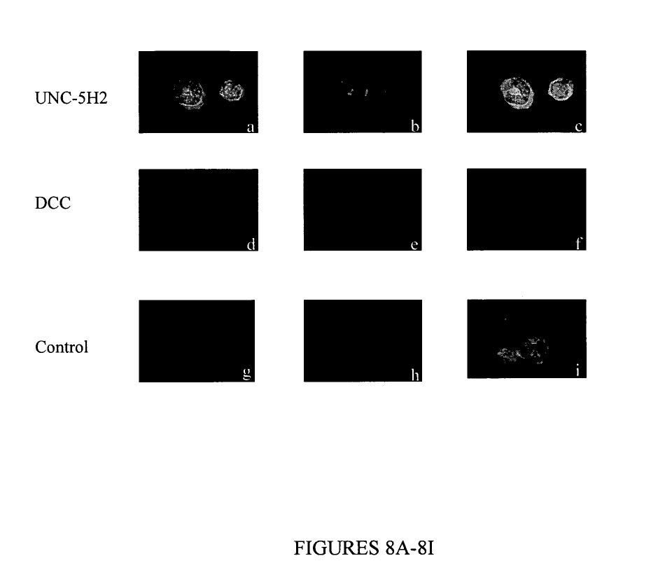

Detection of DCC and UNC5H2 was performed by immunohistochemistry in human monocytes and lymphocytes using a commercially available DCC antibody (Santa Cruz # sc6535) and a rabbit UNC5H2 antiserum generated against an extracellular domain comprising the sequence of: SQAGTDSGSEVLPDS. DCC was not detectable in human leukocytes, monocytes or lymphocytes by immunohistochemistry (FIG. 8) or by quantitative RT-PCR. In contrast, UNC5H2 (red) was detected on the surface of monocytes, granulocutes and lymphocytes (FIG. 8). The UNC5H2 staining was clustered on the cell surface in a pattern consistent with its localization to lipid rafts. Surface localization was confirmed by co-localization with CD45. Nuclear DNA was visualized by Hoechst staining (blue).

### Example 4

### In Vitro and in Vivo Inhibition of Immune Cell Recruitment in the Presence of Netrin 1

The effects of Netrin 1 on fMLP, SDF-1, or IL-8-induced chemotaxis on human peripheral blood monocytes, lymphocytes, and granulocytes were examined. Cell migrations were observed under conditions without chemotactic agents; with chemotactic agents including stromal cell-derived factor (SDF)-1α (10 ng/ml), interleukin-8 (IL-8; 50 ng/ml), and formyl-methionine-leucine-phenylalanine (fLMP; 10 nM) with Netrin 1 alone (0.5 μg/ml) or in the presence of chemotactic agents.

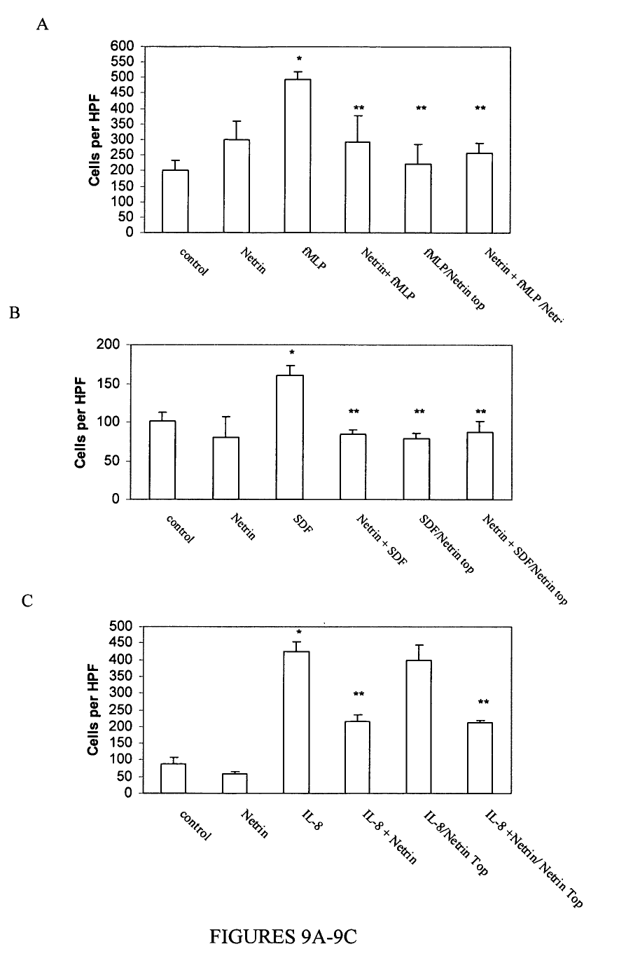

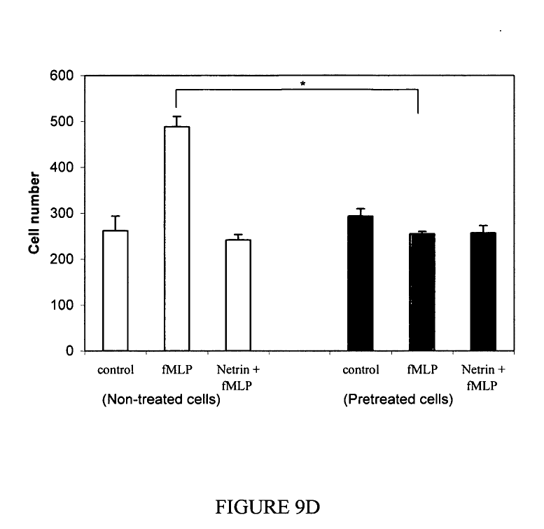

Experiments were initially performed by measuring the migration of cells through the 5 um pores of polyvinylpyrolidone-free polycarbonate filter and counting cells that migrated onto the undersurface. Different chemotactic agents and/or Netrin (28 μl) were placed in the lower wells of a 48-well chemotaxis chamber (Neuroprobe). Cell suspensions (50 μl) were added to the upper wells. The upper and lower wells were separated with a 3 μm or 5 μm pore size polyvinylpyrrolidone-free polycarbonate filter pre-coated with type IV collagen (Sigma). After incubation at 37° C. with 5% CO₂ (30 minutes to 1.5 hours), cells on the top surface of the filter were removed. Cells that have migrated through the undersurface of the filter were fixed, stained, and counted. Migrated cells were expressed as cell number per high-power field. Results of chemotaxis are representative of at least three independent experiments performed in triplicate. Netrin strongly inhibited fMLP-induced chemotaxis of monocytes as depicted in FIG. 9A.

The experiment was repeated using a 96-well assay system where migration of cells into the lower chamber media was measured. Identical results were seen with both techniques. The inhibition of monocyte migration by Netrin 1 was robust, and changing the serum concentrations in the media did not alter this inhibition.

Netrin alone did not alter the baseline cellular migration, even when placed in the upper chamber, where it may exert repulsive forces. The addition of Netrin 1 to either the top (t) or bottom (b) chamber greatly abolished fMLP-induced chemotaxis. Similar results were seen for lymphocytes (SDF-1) and granulocytes (IL-8) (FIG. 9B-C). Thus, Netrin 1 abolished chemotactic responses in all three cell lines (monocytes, lymphocytes, and neutrophils). To rule out a direct interaction between Netrin 1 and fMLP, cells were also pretreated with Netrin 1 for 30 minutes and washed extensively with PBS prior to fMLP exposure (FIG. 9D). This Netrin pretreatment, followed by washout, also abolished fMLP-induced chemotaxis, excluding the possibility of a direct interaction between Netrin 1 and fMLP. Netrin 1 is unlikely to induce toxic effects as in the CNS, Netrin 1 is a survival factor (Liambi, F., et al, EMBO J., 20: 2715-22). However, trypan blue exclusion was not enhanced by Netrin 1 treatment. Together, these results demonstrate that Netrin 1 abolished chemotactic responses of all leukocyte subpopulations and chemokines tested, pointing to its role in inhibiting leukocyte migration.

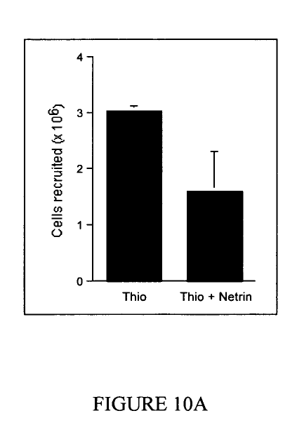

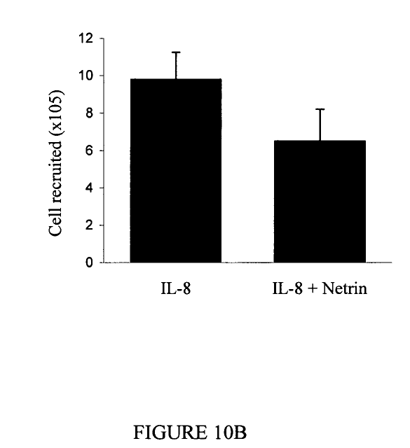

An in vivo peritonitis model was used to determine if recruitment is similarly abolished in vivo. Mice were injected with 3% thioglycollate (Gibco) and cells were collected by peritoneal lavage after 3 hours, counted, and stained with Giemsa stain. All reagents were endotoxin free. Netrin 1 reduced chemotaxis of granulocytes by 50%, as seen in FIG. 10. Thioglycollate alone recruited 74% granulocytes, 18% mononuclear cells, and 8% lymphocytes, whereas thioglycollate in the presence of Netrin 1 recruited 57% granulocytes, 30% mononuclear cells, and 13% lymphocytes. In a similar experiment, mice were injected intraperitoneally with IL-8 and recruited leukocytes were harvested by peritoneal lavage after 4 hours. Netrin 1 reduced the total number of leukocytes recruited to the peritoneum by 48% (p<0.005; see FIG. 10B).

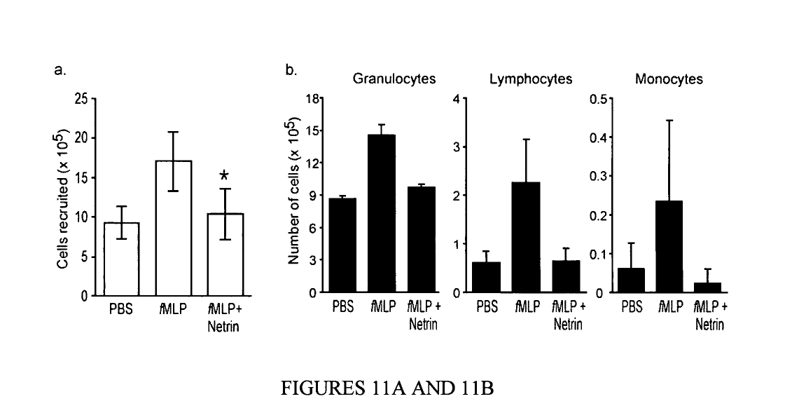

In another similar experiment, mice were injected intraperitoneally with fMLP (10 nM), Netrin 1 (500 ng/ml) or both, and recruited cells were collected by peritoneal lavage after 3 hours, counted and stained with Giemsa stain as previously described. Mice were injected intraperitoneally with 1 ml of PBS+1% BSA, fMLP (10 nM), Netrin 1 (500 ng) or both fMLP+Netrin 1 (500 ng) and recruited leukocytes were harvested by peritoneal lavage after 3 hours (FIG. 11A). fMLP induced a 2-fold increase in leukocytes in the peritoneum that was abrogated in the presence of Netrin 1 (*P<0.005). Injection of Netrin 1 alone was similar to the PBS control (not shown). Analysis of the leukocyte sub-populations in the peritoneum by Giemsa staining revealed that 3 hours after fMLP injection, a predominance of granulocytes was recruited to the peritoneum (FIG. 11B). Netrin 1 did not alter the different leukocyte subpopulations recruited, but similarly inhibited granulocytes, lymphocytes and monocytes. fMLP caused a rapid recruitment of leukocytes to the peritoneum (FIG. 11A), characterized by a predominance of granulocytes (92%) and few monocytes (2%) or lymphocytes (6%) (FIG. 11B). In the additional presence of Netrin 1, the total number of leukocytes recruited to the peritoneum was reduced by 45% (P<0.005) (FIG. 11A). Netrin 1 did not alter the distribution of leukocyte subpopulations recruited by fMLP, but rather reduced the migration of all leukocytes into the peritoneum. Injection of Netrin 1 alone did not induce leukocyte recruitment into the peritoneum and was similar to the PBS control.

### Example 5

### Changes in Netrin 1 Expression During Infection in Vivo

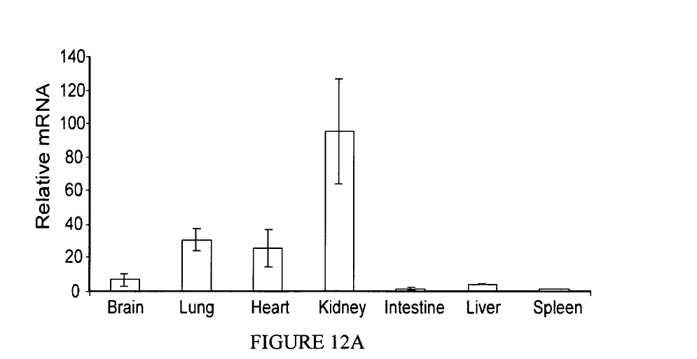

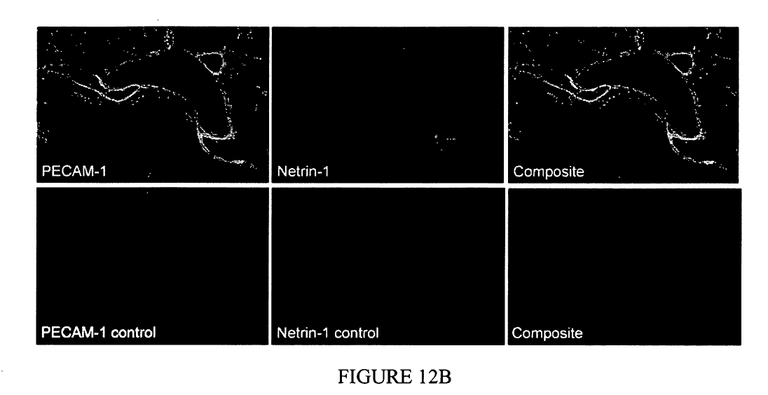

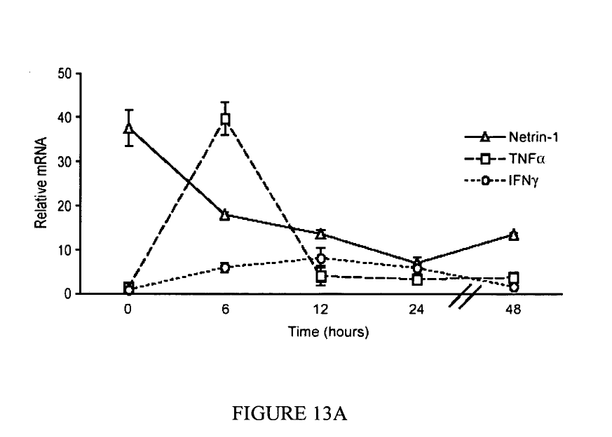

The expression of Netrin 1 in non-CNS tissues (e.g., kidney, liver, spleen and small intestine) was first determined by quantitative RT-PCR using a Bio-Rad iCycler. In addition to brain, strong expression was found in the lung as well as the kidneys, heart (FIG. 12). Lesser expression was found in the liver, intestine and spleen. To define the precise localization of Netrin 1, tissues were fixed in 4% paraformaldehyde and sectioned. Netrin 1 was highly expressed in the lung, with abundant staining detected in the vascular endothelium of large and small blood vessels. This endothelial expression was confirmed by colocalization of Netrin 1 with the endothelial cell marker, PECAM-1. Expression of Netrin 1 in this location is consistent with a role in the regulation of inflammatory cell migration.

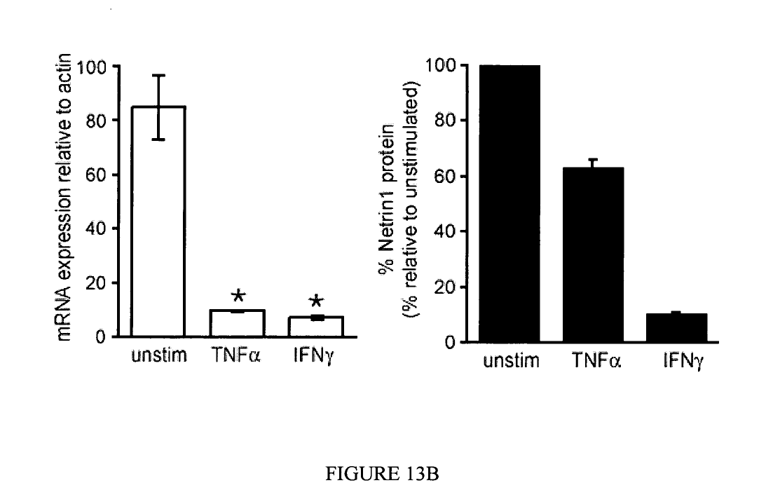

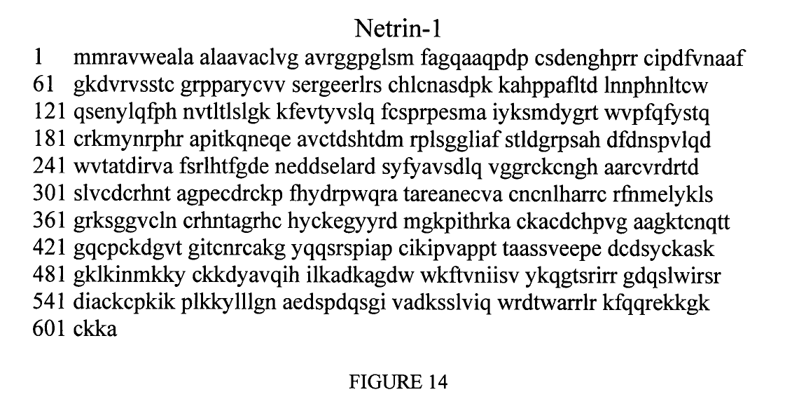

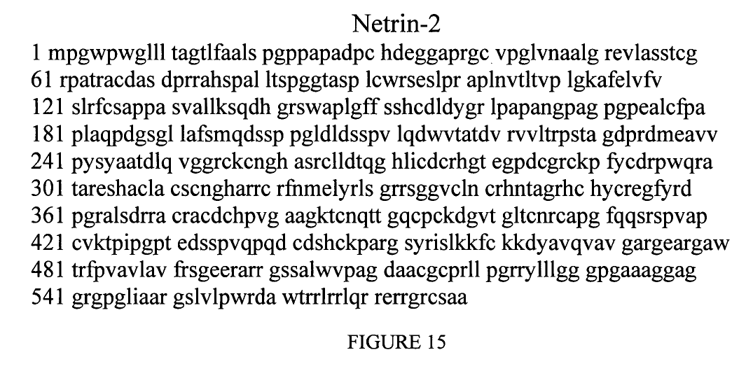

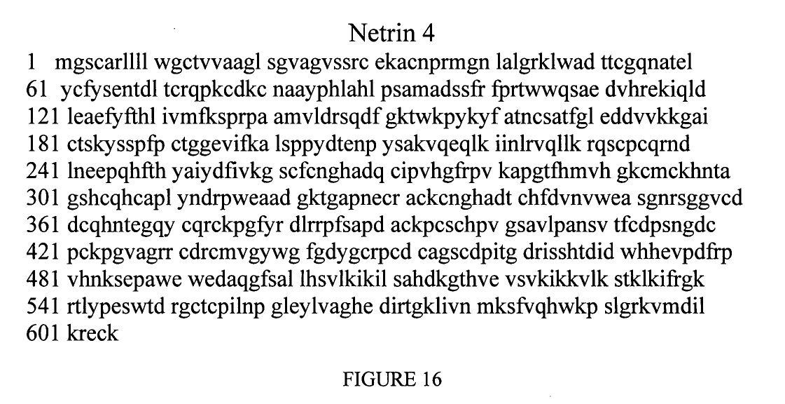

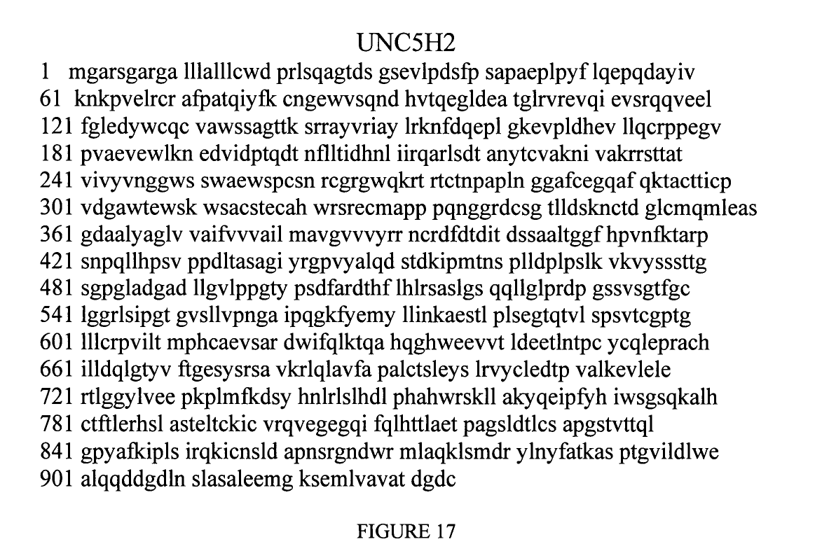

Regulation of leukocyte migration at the endothelium, should be modulated during acute inflammation due to infection. To test this, a mouse model of Staphylococcus aureus infection in which the lung is a site of abscess formation was generated. In mice injected with S. aureus, Netrin 1 expression was rapidly downregulated in the lung (FIG. 13A). At 6 hours post-injection with S. aureus, there was a 2-fold reduction in Netrin 1 mRNA in the lung as determined by quantitative real-time RT-PCR. This reduction in Netrin 1 coincided with a 6-fold increase in IFN-γ and a 27-fold increased in TNF-α mRNA (FIG. 13A). Expression of these cytokines corresponds with the accumulation of leukocytes in the lung. Netrin 1 levels continued to decline until 24 hours post-infection, and this was followed by a trend upward by 48 hours. Thus, in this model of acute inflammation of the lung, Netrin 1 expression is rapidly downregulated during the initial phase of inflammation.

Cytokines such as TNF-α and IFN-γ are major mediators of acute inflammation and were found to have an inverse expression pattern to Netrian-1 during lung abscess formation. To test whether these cytokines could modulate Netrin 1 expression in vascular endothelium, HUVECs were stimulated with TNF-α and IFN-γ. Treatment with these cytokines significantly reduced Netrin 1 mRNA expression in HUVECs (FIG. 13B), suggesting a role for these inflammatory mediators in the regulation of Netrin 1 expression by endothelium.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following numbered claims.

### REFERENCES

Ando, Y., Wajjwalku, W., Kishihara, K., Arai, T., Niimi, N., Hiromatsu, K., Morishima, T., Yoshikai, Y.: “CD4 expression is important but not essential for infection with exogenous mouse mammary tumor virus”. (1996) Immunobiology 195: 376-84.
Anselmo, M. A., Dalvin, S., Prodhan, P., Komatsuzaki, K., Aidlen, J. T., Schnitzer, J. J., Wu, J. Y., Kinane, T. B.: “Slit and Robo: Expression Patterns in Lung Development”. (2003) Gene Expr. Patterns 3(1):13-19.
Ausubel, F. M., Brent, R., Kingston, R., Moore, D., Seidman, J., Struhl, K. 1997. Current Protocols in Molecular Biology. John Wiley and Sons, New York, N.Y.
Becknew, S.: “G-protein activation by chemokines”. In: Horuk, R., ed. Academic Press, (1997) Meth. Enzymol. Vol. 288: 309-326.
Bennett, K. L., Bradshaw, J., Youngman, T., Rodgers, J., Greenfield, B., Aruffo, A., Linsley, P. S.: “Deleted in colorectal carcinoma (DCC) binds heparin via its fifth fibronectin type III domain”. (1997) J. Biol. Chem. 272: 26940-6.
Birnbaumer, L.: “Receptor-to-effector signaling through G proteins: roles for beta gamma dimers as well as alpha subunits”. (1992) Cell 71: 1069-72.
Boes, M., Prodeus, A. P., Schmidt, T., Carroll, M. C., Chen, J.: “A critical role of natural immunoglobulin M in immediate defense against systemic bacterial infection” (1998) J. Exp. Med. 188: 2381-6.
Bokoch, G. M.: “Chemoattractant signaling and leukocyte activation”. (1995) Blood 86: 1649-60.
Cacalano, G., Lee, J., Kikly, K., Ryan, A. M., Pitts-Meek, S., Hultgren, B., Wood, W. I., Moore, M. W.: “Neutrophil and B cell expansion in mice that lack the murine IL-8 receptor homolog.” (1994) Science 265: 682-4.
Chaffin, K. E., Perlmutter, R. M.: “A pertussis toxin-sensitive process controls thymocyte emigration”. (1991) Eur. J. Immunol. 21: 2565-73.
Chan, S. S., Zheng, H., Su, M. W., Wilk, R., Killeen, M. T., Hedgecock, E. M., and Culotti, J. G.: “UNC-40, a C. elegans homolog of DCC (Deleted in Colorectal Cancer), is required in motile cells responding to UNC-6 netrin cues”. (1996) Cell 87: 187-95.
Chickering, D. E., et al., Biotechnol. Bioeng. 52: 96-101.
Coligan, J. E., Kruisbeek, A. M., Margulies, D. H., Shevach, E. M., Strober, W. 1991. Current Protocols in Immunology. John Wiley and Sons, New York, N.Y.
Crockett-Torabi, E., “Selectins and mechanisms of signal transduction”. (1998) J Leukoc Biol. 63(1): 1-14.
Dahlgren, C., and Karlsson, A.: “Respiratory burst in human neutrophils” (1999) J. Immunol. Meth. 232: 3-14.
Dalvin, S., Anselmo, M. A., Prodhan, P., Komatsuzaki, K., Schnitzer, J. J., and Kinane, T. B.: “Expression of Netrin 1 and its two receptors DCC and UNC5H2 in the developing mouse lung”. (2003) Gene Expr. Patterns 3(3): 279-83.
De Vries, L., Zheng, B., Fischer, T., Elenko, E., Farquhar, M. G.: “The regulator of G protein signaling family”. (2000) Ann. Rev. Pharmacol. Toxicol. 40: 235-71.
Diomede, L. Albani, D., Sottocorno, M., Donati, M. B., Bianchi, M., Fruscella, P., Salmona, M.: “In vivo anti-inflammatory effect of statins is mediated by nonsterol mevalonate products” 2001. Arterioscler. Thromb. Vasc. Biol. 21(8): 1327-32.
Ekerdt, R., Muller, B.: “Role of prostanoids in the inflammatory reaction and their therapeutic potential in the skin”. (1992) Arch. Dermatol. Res. 284 Suppl 1: S18-21.
Engelkamp, D.: “Cloning of three mouse Unc5 genes and their expression patterns at mid-gestation”. (2002) Mech. Dev. 118: 191-7.
Forcet, C., Stein, E., Pays, L., Corset, V., Llambi, F., Tessier-Lavigne, M., Mehlen, P.: “Netrin 1-mediated axon outgrowth requires deleted in colorectal cancer-dependent MAPK activation”. (2002) Nature 417: 443-7.
Fraley, R., and Papahadjopoulos, D.: “New generation liposomes: the engineering of an efficient vehicle for intracellular delivery of nucleic acids” (1981) Trends Biochem. Sci. 6: 77-80.
Freshney, R. I., 1987. Animal Cell Culture: A Practical Approach. John Wiley and Sons, New York, N.Y.
Gahmberg, C. G., Valmu, L., Fagerholm, S., Kotovuori, P., Ihanus, E., Tian, L., Pessa-Morikawa, T.: “Leukocyte integrins and inflammation”. (1998) Cell Mol. Life Sci. 54(6): 549-55.
Gait, M. J. 1984. Oligonucleotide Synthesis: A Practical Approach. IRL Press, Oxford, United Kingdom.
Goodman Gilman, A., Hardman, J. G., Limbird, L. E., Molinoff, P. B., Ruddon, R. W. (eds) Goodman & Gilman's The Pharmacological Basis of Therapeutics 9^{th} Edition. (1996) McGraw-Hill, New York.
Gregoriadis, G.: Trends Biotechnol. 3: 235-241.
Hannigan, M. O., Huang, C. K., Wu, D. Q.: “Roles of PI3K in neutrophil function”. (2004) Curr. Topics Microbiol. Immunol. 282: 165-75.
Haribabu, B., Richardson, R. M., Fisher, I., Sozzani, S., Peiper, S. C., Horuk, R., Ali, H., Snyderman, R.: “Regulation of human chemokine receptors CXCR4. Role of phosphorylation in desensitization and internalization”. (1997) J. Biol. Chem. 272: 28726-31.
Harris, R., Sabatelli, L. M., Seeger, M. A.: “Guidance cues at the Drosophila CNS midline: identification and characterization of two Drosophila Netrin/UNC-6 homologs”. (1996) Neuron 17: 217-28.
Hedgecock, E. M., Culotti, J. G., Hall, D. H.: “The unc-5, unc-6, and unc-40 genes guide circumferential migrations of pioneer axons and mesodermal cells on the epidermis in C. elegans”. (1990) Neuron 4: 61-85.
Hedrick, L., Cho, K. R., Fearon, E. R., Wu, T. C., Kinzler, K. W., Vogelstein, B.: “The DCC gene product in cellular differentiation and colorectal tumorigenesis”. (1994) Genes Dev. 8: 1174-83.
Hepler, J. R., Gilman, A. G.: “G proteins”. (1992) Trends Biochem. Sci. 17: 383-7.
Hong, K., Hinck, L., Nishiyama, M., Poo, M. M., Tessier-Lavigne, M., Stein, E.: “A ligand-gated association between cytoplasmic domains of UNC5 and DCC family receptors converts netrin-induced growth cone attraction to repulsion”. (1999) Cell 97: 927-41.
Hopker, V. H., Shewan, D., Tessier-Lavigne, M., Poo, M. M., Holt, C.: “Growth-cone attraction to Netrin 1 is converted to repulsion by laminin-1”. (1999) Nature 401: 69-73.
Keleman, K., Dickson, B. J.: “Short- and long-range repulsion by the Drosophila Unc5 netrin receptor”. (2001) Neuron 32: 605-17.
Kennedy, T. E., Serafini, T., de la Torre, J. R., Tessier-Lavigne, M.: “Netrins are diffusible chemotropic factors for commissural axons in the embryonic spinal cord”. (1994) Cell 78: 425-35.
Kinane, T. B., Kang, I., Chu, A., Chin, S. H., Ercolani, L.: “G alpha(i-2) mediates renal LLC-PK1 growth by a Raf-independent activation of p42/p44 MAP kinase”. (1997) Am. J. Physiol. 272: F273-82.
Kinane, T. B., Komatsuzaki, K., Aleixo, M. D., Sunday, M. E., Ercolani, L.: “Regulation of the G protein Galphai2 by growth and development in fetal airway epithelium”. (1999) Am. J. Respir. Cell Mol. Biol. 20: 35-42.
Kling, D. E., Narra, V., Islam, S., Kinane, T. B., Alessandrini, A., Ercolani, L., Donahoe, P. K., Schnitzer, J. J.: “Decreased mitogen activated protein kinase activities in congenital diaphragmatic hernia-associated pulmonary hypoplasia”. (2001) J. Pediatr. Surg. 36: 1490-6.
Komatsuzaki, K., Murayama, Y., Giambarella, U., Ogata, E., Seino, S., Nishimoto, I.: “A novel system that reports the G-proteins linked to a given receptor: a study of type 3 somatostatin receptor”. (1997) FEBS Lett. 406: 165-70.
Komatsuzaki, K., Terashita, K., Kinane, T. B., Nishimoto, I.: “Somatostatin type V receptor activates c-Jun N-terminal kinases via Galpha(12) family G proteins”. (2001) Biochem. Biophys. Res. Commun. 289: 1211-7.
Komatsuzaki, K., Dalvin, S., Kinane, T. B.: “Modulation of G(ialpha(2)) signaling by the axonal guidance molecule UNC5H2”. (2002) Biochem. Biophys. Res. Commun. 297: 898-905.
Kuby, J. (1998) In: Immunology 3^{rd} Edition W.H. Freeman and Company, New York Langer, R., Brem, H., and Tapper, D.: “Biocompatibility of polymeric delivery systems for macromolecules”. (1981) J. Biomed. Mater. Res. 15(2): 267-77.
Langer, R. (1982) Chem. Tech. 12: 98-105.
Lee, J. S., Hmama, Z., Mui, A., Reiner, N. E.: “Stable gene silencing in human monocytic cell lines using lentiviral-delivered siRNA: Silencing of the p110a isoform of phosphoinositide 3-kinase reveals differential regulation of adherence induced by 1a, 25-dihydroxycholecalciferol and bacterial lipopolysaccharide”. (2003) J. Biol. Chem. 10.1074/jbc.M310638200.
Leonardo, E. D., Hinck, L., Masu, M., Keino-Masu, K., Ackerman, S. L., Tessier-Lavigne, M.: “Vertebrate homologues of C. elegans UNC-5 are candidate netrin receptors”. (1997) Nature 386: 833-8.
Leung-Hagesteijn, C., Spence, A. M., Stern, B. D., Zhou, Y., Su, M. W., Hedgecock, E. M., Culotti, J. G.: “UNC-5, a transmembrane protein with immunoglobulin and thrombospondin type 1 domains, guides cell and pioneer axon migrations in C. elegans”. (1992) Cell 71: 289-99.
Li, Z., Jiang, H., Xie, W., Zhang, Z., Smrcka, A. V., Wu, D.: “Roles of PLC-beta2 and -beta3 and PI3 Kgamma in chemoattractant-mediated signal transduction”. (2000) Science 287: 1046-9.
Livesey, F. J., Hunt, S. P.: “Netrin and netrin receptor expression in the embryonic mammalian nervous system suggests roles in retinal, striatal, nigral, and cerebellar development”. (1997) Mol. Cell. Neurosci. 8: 417-29.
Llambi, F., Causeret, F., Bloch-Gallego, E., Mehlen, P.: “Netrin 1 acts as a survival factor via its receptors UNC5H and DCC”. (2001) EMBO J. 20: 2715-22.
Luster, A. D.: “Chemokines—chemotactic cytokines that mediate inflammation”. (1998) New Engl. J. Med. 338: 436-45.
Mathiowitz, E., Jacob, J. S., Jong, Y. S., Carino, G. P., Chickering, D. E., Chaturvedi, P., Santos, C. A., Vijayaraghavan, K., Montgomery, S., Bassett, M., Morrell, C.: “Biologically erodable microspheres as potential oral drug delivery systems” (1997) Nature 386(6623): 410-4.
Medeiros, L. A., Khan, T., El Khoury, J. B., Pham, C. L., Hatters, D. M., Howlett, G. J., Lopez, R., O'Brien, K. D., Moore, K. J.: “Fibrillar amyloid protein present in atheroma activates CD36-signal transduction”. (2003) J. Biol. Chem. 10.1074/jbc.M311735200.
Merz, D. C., Zheng, H., Killeen, M. T., Krizus, A., Culotti, J. G.: “Multiple signaling mechanisms of the UNC-6/netrin receptors UNC-5 and UNC-40/DCC in vivo”. (2001) Genetics 158: 1071-80.
Miller J., and Calos, M. P. (1987) Gene Transfer Vectors for Mammalian Cells. Cold Spring Harbor Laboratory Press, New York, N.Y.
Moore, K. J., El Khoury, J., Medeiros, L. A., Terada, K., Geula, C., Luster, A. D., Freeman, M. W.: “A CD36-initiated signaling cascade mediates inflammatory effects of beta-amyloid”. (2002) J. Biol. Chem. 277: 47373-9.
Moxham, C. M., Malbon, C. C.: “Insulin action impaired by deficiency of the G-protein subunit Gialpha2”. (1996) Nature 379: 840-4.
Mullis, K. B., 1994. PCR: The Polymerase Chain Reaction. Birkhauser Press, Cambridge, Mass.
Murphy, P. M.: “The molecular biology of leukocyte chemoattractant receptors”. (1994) Ann. Rev. Immunol. 12: 593-633.
Nathan, C. F.: “Neutrophil activation on biological surfaces. Massive secretion of hydrogen peroxide in response to products of macrophages and lymphocytes”. (1987) J Clin Invest. 80(6): 1550-60.
Niwa, S., Totsuka, T., Hayashi, S.: “Inhibitory effect of fluvastatin, an HMG-CoA reductase inhibitor, on the expression of adhesion molecules on human monocyte cell line” 1996. Int. J. Immunopharmacol. 18(11): 669-75.
Ono, S. J., Nakamura, T., Miyazaki, D., Ohbayashi, M., Dawson, M., Toda, M.: “Chemokines: roles in leukocyte development, trafficking, and effector function”. (2003) J. Allergy Clin. Immunol. 111(6): 1185-99.
Otto, D., Frotscher, M., and Unsicker, K.: “Basic fibroblast growth factor and nerve growth factor administered in gel foam rescue medial septal neurons after fimbria formix transection” (1989) J. Neurosci. Res. 22(1): 83-91.
Otto, D., and Unsicker, K.: “Basic FGF reverses chemical and morphological deficits in the nigrostriatal system of MPTP-treated mice” (1990) J. Neurosci. 10(6): 1912-21.
Pace, A. M., Faure, M., Bourne, H. R.: “Gi2-mediated activation of the MAP kinase cascade”. (1995) Mol. Biol. Cell 6: 1685-95.
Pelaia, G., Vatrella, A., Cuda, G., Maselli, R., Marsico, S. A.: “Molecular mechanisms of corticosteroid actions in chronic inflammatory airway diseases” 2003. Life Sci. 0.72(14): 1549-61.
Pettit, E. J., and Fay, F. S.: “Cytosolic free calcium and the cytoskeleton in the control of leukocyte chemotaxis”. (1998) Physiol. Rev. 78: 949-967.
Prodeus, A. P., Zhou, X., Maurer, M., Galli, S. J., Carroll, M. C.: “Impaired mast cell-dependent natural immunity in complement C3-deficient mice”. (1997) Nature 390: 172-5.
Przyborski, S. A., Knowles, B. B., Ackerman, S. L.: “Embryonic phenotype of Unc5h3 mutant mice suggests chemorepulsion during the formation of the rostral cerebellar boundary”. (1998) Development (Supplement) 125: 41-50.
Ramet, M., Manfruelli, P., Pearson, A., Mathey-Prevot, B., Ezekowitz, R. A.: “Functional genomic analysis of phagocytosis and identification of a Drosophila receptor for E. coli”. (2002) Nature 416: 644-8.
Remington, J. P.: (1990) Remington's Pharmaceutical Sciences, 18th edition, Mack Publishing Company, Easton, Pa.
Richmond, A., Mueller, S., Whiter, W. R., Schraw, W.: “C-X-C chemokine receptor desensitization mediated through ligand-enhanced receptor phosphorylation on serine residues”. (1997) In: Horuk, R., ed. Academic Press, Meth. Enzymol. Vol. 288: 3-15.
Ridley, A. J.: “Rho family proteins: coordinating cell responses”. (2001) Trends Cell Biol. 11:471-7.
Ridley, A. J.: “Rho proteins, PI 3-kinases, and monocyte/macrophage motility”. (2001) FEBS Lett. 498: 168-71.
Romano, M., Diomede, L., Sironi, M., Massimiliano, L., Sottocomo, M., Polentarutti, N., Guglielmotti, A., Albani, D., Bruno, A., Fruscella, P., Salmona, M., Vecchi, A., Pinza, M., Mantovani, A.: “Inhibition of monocyte chemotactic protein-1 synthesis by statins”. 2000 Lab. Invest. 80(7): 1095-1100.
Rudolph, U., Finegold, M. J., Rich, S. S., Harriman, G. R., Srinivasan, Y., Brabet, P., Boulay, G., Bradley, A., Bimbaumer, L.: “Ulcerative colitis and adenocarcinoma of the colon in G alpha i2-deficient mice”. (1995) Nat. Genet. 10: 143-50.
Salminen, M., Meyer, B. I., Bober, E., Gruss, P.: “Netrin 1 is required for semicircular canal formation in the mouse inner ear”. (2000) Development 127:13-22.
Sambrook, J. 1989. Molecular Cloning: A Laboratory Manual, second edition, Cold Spring Harbor Laboratory Press, New York, N.Y.
Serafini, T., Kennedy, T. E., Galko, M. J., Mirzayan, C., Jessell, T. M., Tessier-Lavigne, M.: “The netrins define a family of axon outgrowth-promoting proteins homologous to C. elegans UNC-6”. (1994) Cell 78: 409-24.
Serafini, T., Colamarino, S. A., Leonardo, E. D., Wang, H., Beddington, R., Skarnes, W. C., Tessier-Lavigne, M.: “Netrin 1 is required for commissural axon guidance in the developing vertebrate nervous system”. (1996) Cell 87: 1001-14.

Sidman, K. R., Steber, W. D., Schwope, A. D., and Schnaper, G. R.: “Controlled release of macromolecules and pharmaceuticals from synthetic polypeptides based on glutamic acid”. (1983) Biopolymers. 22(1): 547-56.
Spangrude, G. J., Braaten, B. A., Daynes, R. A.: “Molecular mechanisms of lymphocyte extravasation. I. Studies of two selective inhibitors of lymphocyte recirculation”. (1984) J. Immunol. 132: 354-62.
Srinivasan, K., Strickland, P., Valdes, A., Shin, G. C., Hinck, L.: “Netrin 1/neogenin interaction stabilizes multipotent progenitor cap cells during mammary gland morphogenesis”. (2003) Dev. Cell 4: 371-82.
Stein, E., Zou, Y., Poo, M., Tessier-Lavigne, M.: “Binding of DCC by Netrin 1 to mediate axon guidance independent of adenosine A2B receptor activation”. (2001) Science 291: 1976-82.
Tager, A. M., Dufour, J. H., Goodarzi, K., Bercury, S. D., von Andrian, U. H., Luster, A. D.: “BLTR mediates leukotriene B(4)-induced chemotaxis and adhesion and plays a dominant role in eosinophil accumulation in a murine model of peritonitis”. (2000) J. Exp. Med. 192: 439-46.
Takahashi, K., Gordon, J., Liu, H., Sastry, K. N., Epstein, J. E., Motwani, M., Laursen, I., Thiel, S., Jensenius, J. C., Carroll, M., Ezekowitz, R. A.: “Lack of mannose-binding lectin-A enhances survival in a mouse model of acute septic peritonitis”. (2002) Microbes Infect. 4: 773-84.
Takano, T., Fiore, S., Maddox, J. F., Brady, H. R., Petasis, N. A., Serhan, C. N.: “Aspirin-triggered 15-epi-lipoxin A4 (LXA4) and LXA4 stable analogues are potent inhibitors of acute inflammation: evidence for anti-inflammatory receptors”. (1997) J. Exp. Med. 185: 1693-704.
Tessier-Lavigne, M., Goodman, C. S.: “The molecular biology of axon guidance”. (1996) Science 274: 1123-33.
Walsh, F. S., Meiri, K., Doherty, P.: “Cell signalling and CAM-mediated neurite outgrowth”. (1997) Soc. Gen. Physiol. Series 52: 221-6.
Weir, K. 1996. Handbook of Experimental Immunology Blackwell Science, Inc. Boston, Mass.
Weitz-Schmidt, G.: “Statins as anti-inflammatory agents” 2002. Trends Pharm. Sci. 23(10): 482-6.
Weng, Y., Siciliano, S. J., Waldburger, K. E., Sirotina-Meisher, A., Staruch, M. J., Daugherty, B. L., Gould, S. L., Springer, M. S., DeMartino, J. A.: “Binding and functional properties of recombinant and endogenous CXCR3 chemokine receptors”. (1998) J. Biol. Chem. 273: 18288-91.
Wichterman, K. A., Baue, A. E., Chaudry, I. H.: “Sepsis and septic shock—a review of laboratory models and a proposal”. (1980) J. Surg. Res. 29: 189-201.
Wu, J. Y., Feng, L., Park, H. T., Havlioglu, N., Wen, L., Tang, H., Bacon, K. B., Jiang, Z. H., Zhang, X. C., Rao, Y.: “The neuronal repellent Slit inhibits leukocyte chemotaxis induced by chemotactic factors”. (2001) Nature 410: 948-52.
Yin, Y., Sanes, J. R., Miner, J. H.: “Identification and expression of mouse netrin-4”. (2001) Mech. Dev. 96:115-9.
Yoshida, M. Sawada, T., Ishii, H., Gerszten, R. E., Rosenzweig, A., Gimbrone, M. A. Jr, Yasukochi, Y., Numano, F.: “HMG-CoA reductase inhibitor modulates monocyte-endothelial cell interaction under physiological flow conditions in vitro: involvement of Rho GTPase-dependent mechanism” 2001. Arterioscler. Thromb. Vasc. Biol. 21(7): 1165-71.

We claim:

1. A method of modulating inflammatory cell movement in a subject, the method comprising contacting an inflammatory cell undergoing or likely to undergo movement with a Netrin polypeptide in an amount effective to decrease movement of the inflammatory cells to a target site in the subject, thereby modulating inflammatory cell movement in the subject.

2. The method of claim 1, wherein the inflammatory cells comprise leukocytes, lymphocytes, natural killer cells, antigen-presenting cells and endothelial cells.

3. The method of claim 2, wherein the leukocytes comprise neutrophils, basophils, mast cells, eosinophils, monocytes, and macrophages.

4. The method of claim 2, wherein the lymphocytes comprise B-lymphocytes and T-lymphocytes.

5. The method of claim 2, wherein the antigen-presenting cells comprise dendritic cells and stromal cells.

6. The method of claim 1, wherein the contacting occurs in vivo in a subject having or at risk of having an adverse immune response comprising inflammatory cell movement to a target site.

7. The method of claim 6, wherein the subject is a human.

8. The method of claim 6, wherein the adverse immune response is an inflammatory response.

9. The method of claim 8, wherein the inflammatory response comprises a disorder selected from the group consisting of acute hemorrhagic leukoencephalitis, acute infantile hemorrhagic edema, allergy, appendicitis, asthma, atherosclerosis, atrophic gastritis, atrophic rhinitis, Barrett's esophagus, blepharitis, Bowenoid papulosis, bronchiolitis obliterans organizing pneumonitis, bronchiectatus, cancrum oris, Celiac disease, cervicitis, cholangitis, cholesterol granuloma, chronic interstitial nephritis, colitis, colonic diverticulitis, conjunctivitis, contact dermatitis, Curling's ulcers, Cushing's ulcers, cystitis, dacryocystitis, De Quervain's tenosynovitis, eczema, pleural empyema, endocarditis, endogenous lipoid pneumonia, endophthalmitis, epidydymo-orchitis, Favre-Racouchot syndrome, folliculitis, Fuch's heterochromic cyclitis, gangrene, giant cell granuloma, giant papillary conjunctivitis, gingivitis, inflammatory bowel disease, Jarisch-Herxheimer reaction, laryngeal granuloma, lymphocytic interstitial pneumonia, mastitis, meningoencephalitis, mesenteric adenitis, Mondor's disease of the breast, myositis, myringitis bullosa, necrotizing sialometaplasia, esophagitis, optic neuritis, osteitis, osteitis fibrosa cystica, osteitis pubis, pancreatitis, panniculitis, parotitis, Parsonage-Aldren-Turner syndrome, pericarditis, pericoronitis, perihepatitis, periodontitis, periostitis, peritonitis, phyrangitis, phlegmonous gastritis, phlyctenular keratoconjunctivitis, pleuritis, posthitis, post-streptococcal glomerulonephritis, proctitis, pseudolymphoma, pulpitis, pyrophosphate arthropathy, radiculitis, reactive arthropathy, Reiter's syndrome, Riehl's melanosis, sacroilitis, scleritis, sinusistis, sterile pneumonitis, Stevens-Johnson syndrome, synovitis, tenosynovitis, thrombophlebitis, thyroiditis, Tietze's costochondritis, toxic megacolon, thrichostasis spinulosa, typhlitis, urate crystal arthropathy, vasculitis, and xanthogranulomatous pyelonephritis.

10. The method of claim 6, wherein the adverse immune response is an autoimmune response.

11. The method of claim 10, wherein the autoimmune response comprises a disorder selected from the group consisting of acquired factor VIII deficiency, acquired generalized lipodystrophy, alopecia greata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyly-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

12. The method of claim 1, wherein the Netrin polypeptide is selected from the group consisting of Netrin 1, Netrin 2, Netrin 4, a homolog of Netrin 1, Netrin 2 or Netrin 4 and an allelic variant of Netrin 1, Netrin 2 or Netrin 4.

13. The method of claim 12, wherein the homolog is 60%, 85%, or 95% homologous to Netrin 1, Netrin 2 and Netrin 4.

14. The method of claim 1, wherein the decrease in movement of the inflammatory cells is measured by changes in inflammatory cell morphology, changes in tissue or organ morphology, changes in inflammatory cell number, changes in gene expression, changes in protein expression, changes in levels of reactive oxygen species, changes in calcium levels, or changes in cAMP levels.

15. The method of claim 14, wherein the changes in inflammatory cell morphology are measured by microscopy or immunocytochemistry.

16. The method of claim 14, wherein the changes in inflammatory cell number are measured by flow cytometry.

17. The method of claim 14, wherein the changes in gene expression are measured by microarray analysis, Northern blotting, in situ hybridization, or RT-PCR.

18. The method of claim 14, wherein the changes in protein expression are measured by Western blotting, immunocytochemistry, colorimetric assay, or ELISA.

19. A method of decreasing inflammatory cell chemotaxis, the method comprising contacting inflammatory cells undergoing or likely to undergo chemotaxis with a Netrin polypeptide in an amount effective to decrease chemotaxic signaling in the inflammatory cells, thereby decreasing inflammatory cell chemotaxis.

20. The method of claim 19, wherein the inflammatory cells comprise leukocytes, lymphocytes, natural killer cells, antigen-presenting cells and endothelial cells.

21. The method of claim 20, wherein the leukocytes comprise neutrophils, basophils, mast cells, eosinophils, monocytes and macrophages.

22. The method of claim 20, wherein the lymphocytes comprise B-lymphocytes and T-lymphocytes.

23. The method of claim 20, wherein the antigen-presenting cells comprise dendritic cells and stromal cells.

24. The method of claim 19, wherein the contacting occurs in vivo in a subject having or at risk of having an adverse immune response comprising chemotaxis of inflammatory cells.

25. The method of claim 24, wherein the subject is a human.

26. The method of claim 24, wherein the adverse immune response is an inflammatory response.

27. The method of claim 26, wherein the inflammatory response comprises a disorder selected from the group consisting of acute hemorrhagic leukoencephalitis, acute infantile hemorrhagic edema, allergy, appendicitis, asthma, atherosclerosis, atrophic gastritis, atrophic rhinitis, Barrett's esophagus, blepharitis, Bowenoid papulosis, bronchiolitis obliterans organizing pneumonitis, bronchiectatus, cancrum oris, Celiac disease, cervicitis, cholangitis, cholesterol granuloma, chronic interstitial nephritis, colitis, colonic diverticulitis, conjunctivitis, contact dermatitis, Curling's ulcers, Cushing's ulcers, cystitis, dacryocystitis, De Quervain's tenosynovitis, eczema, pleural empyema, endocarditis, endogenous lipoid pneumonia, endophthalmitis, epidydymo-orchitis, Favre-Racouchot syndrome, folliculitis, Fuch's heterochromic cyclitis, gangrene, giant cell granuloma, giant papillary conjunctivitis, gingivitis, inflammatory bowel disease, Jarisch-Herxheimer reaction, laryngeal granuloma, lymphocytic interstitial pneumonia, mastitis, meningoencephalitis, mesenteric adenitis, Mondor's disease of the breast, myositis, myringitis bullosa, necrotizing sialometaplasia, esophagitis, optic neuritis, osteitis, osteitis fibrosa cystica, osteitis pubis, pancreatitis, panniculitis, parotitis, Parsonage-Aldren-Turner syndrome, pericarditis, pericoronitis, perihepatitis, periodontitis, periostitis, peritonitis, phyrangitis, phlegmonous gastritis, phlyctenular keratoconjunctivitis, pleuritis, posthitis, post-streptococcal glomerulonephritis, proctitis, pseudolymphoma, pulpitis, pyrophosphate arthropathy, radiculitis, reactive arthropathy, Reiter's syndrome, Riehl's melanosis, sacroilitis, scleritis, sinusistis, sterile pneumonitis, Stevens-Johnson syndrome, synovitis, tenosyriovitis, thrombophlebitis, thyroiditis, Tietze's costochondritis, toxic megacolon, thrichostasis spinulosa, typhlitis, urate crystal arthropathy, vasculitis, and xanthogranulomatous pyelonephritis.

28. The method of claim 24, wherein the adverse immune response is an autoimmune response.

29. The method of claim 28, wherein the autoimmune response is a disorder selected from the group consisting of acquired factor VIII deficiency, acquired generalized lipodystrophy, alopecia greata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyly-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

30. The method of claim 19, wherein the Netrin polypeptide is selected from the group consisting of Netrin 1, Netrin 2, Netrin 4, a homolog of Netrin 1, Netrin 2 or Netrin 4 and an allelic variant of Netrin 1, Netrin 2 or Netrin 4.

31. The method of claim 30, wherein the homolog is 60%, 85%, or 95% homologous to Netrin 1, Netrin 2 and Netrin 4.

32. The method of claim 19, wherein the decrease in chemotaxis of the inflammatory cells is measured by changes in inflammatory cell morphology, changes in tissue or organ morphology, changes in tissue or organ morphology, changes in inflammatory cell number, changes in gene expression, changes in protein expression, changes in levels of reactive oxygen species, changes in calcium levels, or changes in cAMP levels.

33. The method of claim 32, wherein the changes in inflammatory cell morphology are measured by microscopy or immunocytochemistry.

34. The method of claim 32, wherein the changes in inflammatory cell number is measured by flow cytometry.

35. The method of claim 32, wherein the changes in gene expression are measured by microarray analysis, Northern blotting, in situ hybridization, or RT-PCR.

36. The method of claim 32, wherein the changes in protein expression are measured by Western blotting, immunocytochemistry, colorimetric assay, or ELISA.

37. A method of treating inflamed tissues in a subject, comprising administering to a subject having at least one inflamed tissue a Netrin polypeptide that decreases inflammatory cell movement in an amount effective to decrease accumulation of inflammatory cells in the tissue, thereby treating the inflamed tissues in the subject.

38. The method of claim 37, wherein the inflamed tissue comprises inflammatory cells.

39. The method of claim 38, wherein the inflammatory cells comprise leukocytes, lymphocytes, natural killer cells, antigen-presenting cells and endothelial cells.

40. The method of claim 39, wherein the leukocytes comprise neutrophils, eosinophils, mast cells, basophils, monocytes, and macrophages.

41. The method of claim 39, wherein the lymphocytes comprise B-lymphocytes and T-lymphocytes.

42. The method of claim 39, wherein the antigen-presenting cells comprise dendritic cells and stromal cells.

43. The method of claim 37, wherein the subject is a human.

44. The method of claim 37, wherein the Netrin polypeptide is selected from the group consisting of Netrin 1, Netrin 2, Netrin 4, a homolog of Netrin 1, Netrin 2 or Netrin 4 and an allelic variant of Netrin 1, Netrin 2 or Netrin 4.

45. The method of claim 44, wherein the homolog is 60%, 85%, or 95% homologous to Netrin 1, Netrin 2 and Netrin 4.

46. The method of claim 37, wherein treating the inflamed tissue further comprises drug therapy.

47. The method of claim 46, wherein the drug is selected from any member of the group consisting of antihistamines, cytokine antagonists, non-steroidal anti-inflammatory agents, eicosanoid receptor inhibitors, monoclonal antibodies, 3-hydroxy-methyl-glutaryl-coenzyme A (HMG-CoA) reductase inhibitors, corticosteroids, and other cytokines.

48. The method of claim 37, wherin the inflamed tissue is a tissue of the peripheral nervous system, the central nervous system, skin, appendix, gastrointestinal tract, respiratory system, eye, reproductive system, gums, liver, renal system, cardiovascular system, breast, lymphatic system, muscle, ear, endocrine system, exocrine system, skeletal system or is a connective tissue.

49. A kit for modulating inflammatory cell movement in a subject comprising a Netrin polypeptide that decreases inflammatory cell movement and instructions for using the Netrin polypeptide to modulate inflammatory cell movement in the subject in accordance with the method of claim 1.

50. A kit for decreasing inflammatory cell chemotaxis comprising an anti-chemotactic Netrin polypeptide and instructions for using the anti-chemotactic Netrin polypeptide to decrease chemotaxic signaling in the inflammatory cells in accordance with the method of claim 19.

51. A kit for treating inflamed tissues in a subject comprising a Netrin polypeptide that decreases inflammatory cell movement and instructions for using the Netrin polypeptide to decrease accumulation of inflammatory cells in the tissue of the subject in accordance with the method of claim 37.

52. A method of screening compositions for Netrin functional activity comprising the steps of:
a) contacting control cells with a Netrin polypeptide and measuring a physiologic effect of the control cells;
b) contacting test cells that do not express a Netrin polypeptide with a test compound suspected of modulating inflammatory cell migration and measuring a physiologic effect of the test cells; and
c) comparing the physiologic effect of the test compound to the physiologic effect of the Netrin polypeptide to identify Netrin functional activity of the test compound.

53. The method of claim 52, wherein the test compound is a non-protein analog of a Netrin polypeptide.

54. The method of claim 52, wherein the physiologic effect of the test compound is measured by changes in inflammatory cell morphology, changes in tissue or organ morphology, changes in tissue or organ morphology, changes in inflammatory cell number, changes in gene expression, changes in protein expression, changes in levels of reactive oxygen species, changes in calcium levels, or changes in cAMP levels.

55. The method of claim 54, wherein the changes in inflammatory cell morphology are measured by microscopy or immunocytochemistry.

56. The method of claim 54, wherein the changes in inflammatory cell number is measured by flow cytometry.

57. The method of claim 54, wherein the changes in gene expression are measured by microarray analysis, Northern blotting, in situ hybridization, or RT-PCR.

58. The method of claim 54, wherein the changes in protein expression are measured by Western blotting, immunocytochemistry, colorimetric assay, or ELISA.

59. A method for identifying an agent that modulates the activity of a Netrin receptor, the method comprising: contacting the Netrin receptor with a test compound; and evaluating an activity of the Netrin receptor, wherein a change in activity relative to a reference value is an indication that the compound is an agent that modulates the receptor.

60. The method of claim 59, wherein the test compound is a non-protein analog of a Netrin polypeptide.

61. The method of claim 59, wherein the reference value comprises changes in inflammatory cell morphology, changes in tissue or organ morphology, changes in tissue or organ morphology, changes in inflammatory cell number, changes in gene expression, changes in protein expression, changes in levels of reactive oxygen species, changes in calcium levels, or changes in cAMP levels.

62. The method of claim 61, wherein the changes in inflammatory cell morphology are measured by microscopy or immunocytochemistry.

63. The method of claim 61, wherein the changes in inflammatory cell number is measured by flow cytometry.

64. The method of claim 61, wherein the changes in gene expression are measured by microarray analysis, Northern blotting, in situ hybridization, or RT-PCR.

65. The method of claim 61, wherein the changes in protein expression are measured by Western blotting, immunocytochemistry, colorimetric assay, or ELISA.

66. A method for identifying an agent useful in the treatment of a disorder related to Netrin receptor modulation, the method comprising: contacting the Netrin receptor with a test compound; and evaluating an activity of the Netrin receptor, wherein a change in activity relative to a reference value is an indication that the test compound is an agent useful in a disorder related to Netrin receptor modulation.

67. The method of claim 66, wherein the Netrin receptor is UNC5H2, a homolog of UNC5H2 or an allelic variant of UNC5H2.

68. A method for treating a subject having a disorder related to Netrin receptor modulation, the method comprising: identifying an agent that selectively binds to a Netrin receptor, and administering to a subject in need of such treatment a pharmaceutical composition comprising the agent which is selective for a Netrin receptor.

69. The method of claim 68, wherein the Netrin receptor is UNC5H2, a homolog of UNC5H2 or an allelic variant of UNC5H2.

70. A method of identifying a Netrin receptor comprising the steps of:
a) contacting test cells that express a Netrin receptor with a Netrin polypeptide and measuring a physiologic effect of the test cells; and
b) contacting test cells that express a receptor suspected of modulating inflammatory cell migration with a Netrin polypeptide and measuring a physiologic effect of the test cells; and
c) comparing the physiologic effect of the test cells to the physiologic effect of the control cells to identify a Netrin receptor; and
d) isolating the Netrin receptor.


SEQUENCE LISTING (6 sequences)

SEQ ID NO: 1 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRTDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 2 - protein, 580 aa, Homo sapiens
MPGWPWGLLLTAGTLFAALSPGPPAPADPCHDEGGAPRGCVPGLVNAALGREVLASSTCGRPATRACDASDPRRAHSPALLTSPGGTASPLCWRSESLPRAPLNVTLTVPLGKAFELVFVSLRFCSAPPASVALLKSQDHGRSWAPLGFFSSHCDLDYGRLPAPANGPAGPGPEALCFPAPLAQPDGSGLLAFSMQDSSPPGLDLDSSPVLQDWVTATDVRVVLTRPSTAGDPRDMEAVVPYSYAATDLQVGGRCKCNGHASRCLLDTQGHLICDCRHGTEGPDCGRCKPFYCDRPWQRATARESHACLACSCNGHARRCRFNMELYRLSGRRSGGVCLNCRHNTAGRHCHYCREGFYRDPGRALSDRRACRACDCHPVGAAGKTCNQTTGQCPCKDGVTGLTCNRCAPGFQQSRSPVAPCVKTPIPGPTEDSSPVQPQDCDSHCKPARGSYRISLKKFCKKDYAVQVAVGARGEARGAWTRFPVAVLAVFRSGEERARRGSSALWVPAGDAACGCPRLLPGRRYLLLGGGPGAAAGGAGGRGPGLIAARGSLVLPWRDAWTRRLRRLQRRERRGRCSAA

SEQ ID NO: 3 - protein, 605 aa, Homo sapiens [sequence omitted]

SEQ ID NO: 4 - protein, 934 aa, Homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQMLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 5 - protein, 15 aa, Artificial Sequence
  Description of Artificial Sequence Synthetic linker peptide
GGGGSGGGGSGGGGS

SEQ ID NO: 6 - protein, 15 aa, Artificial Sequence
  Description of Artificial Sequence Synthetic peptide
SQAGTDSGSEVLPDS